SEQUENCE LISTING (21 sequences)

SEQ ID NO: 1 - protein, 20 aa, synthetic construct
GGGGSGGGGSGGGGSGGGGS

SEQ ID NO: 2 - protein, 248 aa, synthetic construct
DIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSS

SEQ ID NO: 3 - protein, 491 aa, synthetic construct
DKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQ

SEQ ID NO: 4 - protein, 511 aa, synthetic construct
DQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGTPSFFRNVVWLIKKNDAYPTIKISYNNTNREDLLIMWGIHHSNNAEEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPVGAINSSMPFHNIHPLTIGECPKYVKSNKLVLATGLRNSPQGETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQ

SEQ ID NO: 5 - protein, 507 aa, synthetic construct
DKICLGHHAVSNGTKVNTLTERGVEVVNATETVERTNTPRICSKGKRTVDLGQCGLLGTITGPPQCDQFLEFSADLIIERREGSDVCYPGKFVNEEALRQILRESGGIDKEPMGFTYNGIRTNGVTSACRRSGSSFYAEMKWLLSNTDNAAFPQMTKSYKNTKESPAIIVWGIHHSVSTAEQTKLYGSGNKLVTVGSSNYQQSFVPSPGARPQVNGQSGRIDFHWLILNPNDTVTFSFNGAFIAPDRASFLRGKSMGIQSRVQVDANCEGDCYHSGGTIISNLPFQNIDSRAVGKCPRYVKQRSLLLATGMKNVPEVPKGRGLFGAIAGFIENGWEGLIDGWYGFRHQNAQGEGTAADYKSTQSAIDQITGKLNRLIAKTNQQFKLIDNEFNEVEKQIGNVINWTRDSITEVWSYNAELLVAMENQHTIDLADSEMDKLYERVKRQLRENAEEDGTGCFEIFHKCDDDCMASIRNNTYDHRKYREEAMQNRIQIDPVKLSSGYKDVI

SEQ ID NO: 6 - protein, 29 aa, synthetic construct
GSGYIPEAPRDGQAYVRKDGEWVLLSTFL

SEQ ID NO: 7 - protein, 520 aa, synthetic construct
DKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFL

SEQ ID NO: 8 - protein, 5 aa, synthetic construct
GSGSG

SEQ ID NO: 9 - protein, 4 aa, synthetic construct
EPEA

SEQ ID NO: 10 - protein, 14 aa, synthetic construct
GKPIPNPLLGLDST

SEQ ID NO: 11 - protein, 18 aa, synthetic construct
MKLCILLAVVAFVGLSLG

SEQ ID NO: 12 - protein, 822 aa, synthetic construct
MKLCILLAVVAFVGLSLGGTGDKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFLLIKGSGSGAAADIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSSSRGKPIPNPLLGLDSTHHHHHH

SEQ ID NO: 13 - protein, 564 aa, synthetic construct
MKLCILLAVVAFVGLSLGGTGDKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFLLIKGKPIPNPLLGLDSTHHHHHH

SEQ ID NO: 14 - DNA, 2466 bp, synthetic construct
  source 1..2466: mol_type = other DNA
atgaagttatgcatattactggccgtcgtggcctttgttggcctctcgctcgggggtaccggggataagatctgcatcggccaccagagcaccaacagcaccgagaccgtggataccctgaccgagaccaacgtgccagtgacccacgccaaggagctgctgcacaccgagcacaacggaatgctgtgcgccaccaatctgggccaccccctgatcctggatacctgcaccatcgagggcctgatctacggcaaccccagctgcgatctgctgctgggaggacgcgagtggtcctacattgtggagcgccccagcgccgtgaacggaacctgctatccaggcaacgtggagaacctggaggagctgcgcaccctgttcagcagctcgagcagctaccagcgcatccagatcttccccgataccatctggaacgtgacctacaccggcaccagcaagagctgcagcgatagcttctaccgcaacatgcgctggctgacccagaagtccggcctgtacccagtgcaggatgcccagtacaccaacaatcgcggcaaggacatcctgttcgtgtggggcatccaccaccccccaaccgataccgcccagaccaatctgtacacccgcaccgataccaccaccagcgtgaccaccgagaatctggatcgcaccttcaagcccgtgatcggcccacgcccactcgtgaatggactgatcggccgcatcaactactattggagcgtgctgaagcccggccagaccctgcgcgtgcgcagcaatggaaatctgatcgccccgtggtacggccacgtgctgagcggagagagccacggccgcattctgaagaccgatctgaacagcggcaactgcgtggtgcagtgccagaccgagaagggcggcctgaatagcaccctgcccttccacaacatctcgaagtacgccttcggaaactgccccaagtacatcggcgtgaagtccctgaagctggccatcggcctgcgcaatgtgccagcccgcagtagtcgcggactgttcggagccattgccggcttcattgagggcggctggccaggactggtggccggatggtacggattccagcacagcaacgatcagggcgtgggaatggccgccgatcgcgatagtacccagaaggccgtggataagatcacctccaaagtgaacaacatcgtggacaagatgaacaagcagtacgagatcatcgaccacgagttcagcgaggtggagacccgcctgaacatgatcaacaacaagatcgacgaccagatccaggatgtgtgggcctacaacgccgagctgctggtgctgctggagaaccagaagaccctggacgagcacgatgccaacgtgaacaatctgtataacaaagtgaagcgcgccctgggcagcaacgccatggaggatggaaagggatgcttcgagctgtaccacaagtgcgacgatcagtgcatggagaccatccgcaacggcacctacaaccgccgcaagtacaaggaggagagccgcctggagcgccagggcagcggctacatcccagaggccccacgcgacggacaggcctatgtgcgcaaggatggcgagtgggtgctgctgagcaccttcctgttaattaagggttctggctctggtgcggccgcggatatcgtgatgacccagtcgccaagcagtctggctgtgtccgtgggacagaaagtgaccatgagctgcaccagcagccaggtgctgctgcacagccccaaccagaagaattacctggcctggtatcagcagaagcccggccaaagtccgaagctgctggtctactttgccagcacacgcgagagcggagtgccagatcgttttaccggaagcggcagcggcaccgatttcaccctgacaattagtagcgtgcaggccgaggatctggccgtgtattactgccagcagcactacagcaccccgctgacatttggcgccggaacgaagctggaactgaaaggcggaggtggtagtggtggcggaggatcaggtggtggtggttctggcggtggtggaagtgaagtgcaactgcagcagagcggcccagagctggtcaaaccaggtgccagcgtgaagatcagctgcaaggccagcggatacaccttcaccgattactacatcaactgggtcaagcagagccacggcaagagcctggaatggatcggcgatatcaaccccaccaacggcgatagcacctacagccagaagttcaagggcaaagccacgctgaccgtggataagagtagcagcaccgcctacatggaactgcgcagcctgacaagcgaagtgtccgccgtgtactattgcgcccgtgattacgccatggattactggggacagggcaccagtgtgaccgttagtagttctagaggtaagcctatccctaaccctctcctcggtctcgattctacgcatcatcaccatcaccat

SEQ ID NO: 15 - DNA, 1692 bp, synthetic construct
  source 1..1692: mol_type = other DNA
atgaagttatgcatattactggccgtcgtggcctttgttggcctctcgctcgggggtaccggggataagatctgcatcggccaccagagcaccaacagcaccgagaccgtggataccctgaccgagaccaacgtgccagtgacccacgccaaggagctgctgcacaccgagcacaacggaatgctgtgcgccaccaatctgggccaccccctgatcctggatacctgcaccatcgagggcctgatctacggcaaccccagctgcgatctgctgctgggaggacgcgagtggtcctacattgtggagcgccccagcgccgtgaacggaacctgctatccaggcaacgtggagaacctggaggagctgcgcaccctgttcagcagctcgagcagctaccagcgcatccagatcttccccgataccatctggaacgtgacctacaccggcaccagcaagagctgcagcgatagcttctaccgcaacatgcgctggctgacccagaagtccggcctgtacccagtgcaggatgcccagtacaccaacaatcgcggcaaggacatcctgttcgtgtggggcatccaccaccccccaaccgataccgcccagaccaatctgtacacccgcaccgataccaccaccagcgtgaccaccgagaatctggatcgcaccttcaagcccgtgatcggcccacgcccactcgtgaatggactgatcggccgcatcaactactattggagcgtgctgaagcccggccagaccctgcgcgtgcgcagcaatggaaatctgatcgccccgtggtacggccacgtgctgagcggagagagccacggccgcattctgaagaccgatctgaacagcggcaactgcgtggtgcagtgccagaccgagaagggcggcctgaatagcaccctgcccttccacaacatctcgaagtacgccttcggaaactgccccaagtacatcggcgtgaagtccctgaagctggccatcggcctgcgcaatgtgccagcccgcagtagtcgcggactgttcggagccattgccggcttcattgagggcggctggccaggactggtggccggatggtacggattccagcacagcaacgatcagggcgtgggaatggccgccgatcgcgatagtacccagaaggccgtggataagatcacctccaaagtgaacaacatcgtggacaagatgaacaagcagtacgagatcatcgaccacgagttcagcgaggtggagacccgcctgaacatgatcaacaacaagatcgacgaccagatccaggatgtgtgggcctacaacgccgagctgctggtgctgctggagaaccagaagaccctggacgagcacgatgccaacgtgaacaatctgtataacaaagtgaagcgcgccctgggcagcaacgccatggaggatggaaagggatgcttcgagctgtaccacaagtgcgacgatcagtgcatggagaccatccgcaacggcacctacaaccgccgcaagtacaaggaggagagccgcctggagcgccagggcagcggctacatcccagaggccccacgcgacggacaggcctatgtgcgcaaggatggcgagtgggtgctgctgagcaccttcctgttaattaagggtaagcctatccctaaccctctcctcggtctcgattctacgcatcatcaccatcaccat

SEQ ID NO: 16 - protein, 246 aa, synthetic construct
EVQLQQSGPELVKPGASVKMSCKASGYTFTNYVLHWVKQKPGQGLEWIGYINPYNDGTKFNEKFKGKATLTSDTSSSTAFMELSSLTSEDSAVYYCARGDNLRPYYFDYWGQGTTLTVSSGGGGSGGGGSGGGGSGGGGSQIVLTHSPAIMSASPGEKVTMTCSASSSVSFMYWYQQKPGSSPRLLLYDTSSLSSGVPVRFSGSGSGTSYSLTISRMEAEDAATYYCQQWSRYPPTFGGGTKLEIK

SEQ ID NO: 17 - protein, 247 aa, synthetic construct
EIVLTQSPALMAASPGEKVTITCSVSSSISSGNFHWYQQKSGTSPKLWIYGTSNLASGVPVRFSGSGSGTSYSLTISSMEAEDAATYYCQQWSSYPFTFGSGTKLEIKGGGGSGGGGSGGGGSGGGGSEVQLVESGGDLVKPGGSLKLSCAASGFTFSSYGMSWVRQTPDKRLEWVATISSGGSYTYYPDSVKGRFTISRDNAKNILYLQMSSLKSEDTAMYYCARLSTWDWYFDVWGTGTTVTVSS

SEQ ID NO: 18 - protein, 470 aa, synthetic construct
ALDGRPLAAAGIVVTGDKAVNVYTSSQTGSIIVKLLPNMPKDKEACARAPLEAYNRTLTTLLTPLGDSIRKIQGSVATSGSGSGSRFIGAVIGSIALGVATAAQITAAAALIQANQNAANILRLKESIAATNEAVHEVTNGLSQLSVAVGKMQQFVNDQFNNTARELDCIKITQQVGVELNLYLTELTTVFGPQITSPALTQLTIQALYNLAGGNMDYLLTKLGVGNNQLSSLIGSGLITGYPILYDSQTQLLGIQVNLPSVGNLNNMRATYLETLSVSTTKGFASALVPKVVTQVGSVIEELDTSYCIESDLDLYCTRIVTFPMSPGIYSCLSGNTSACMYSKTEGALTTPYMALKGSVIANCKITTCRCADPPGIISQNYGEAVSLIDRHSCNVLSLDGITLRLSGEFDATYLKNISILDSQVIVTGNLDISTELGNVNNSISNALDKLTESNSKLDKVNVRLTSTSA

SEQ ID NO: 19 - protein, 525 aa, synthetic construct
SMGTSTPRDLTGISIAISKTEDKVTSLLSSSQDVIDRIYKQVALESPLALLNTESIIMNAITSLSYQINGAANNSGCGAPVHDPDYIGGIGKELIVDDTSDVTSFYPSAYQEHLNFIPAPTTGSGCTRIPSFDMSTTHYCYTHNVILSGCRDHSHSHQYLALGVLRTSATGKVFFSTLRSINLDDTQNRKSCSVSATPLGCDILCSKVTETEEEDYKSVTPTSMVHGRLGFDGQYHEKDLDTTALFKDWVANYPGVGGGSFVDERVWFPVYGGLKPNSPSDTAQEGKYVIYKRYNDTCPDEQDYQIRMAKSSYKPGRFGGKRVQQAILSIKVSTSLGEDPMLTIPPNTITLMGAEGRILTVGTSHFLYQRGSSYFSPALLYPMTISNKTATLHSPYTFNAFTRPGSVPCQASARCPNSCITGVYTDPYPLIFHRNHTLRGVFGTMLDDGQARLNPVSAVFDDISRSRVTRVSSSSTKAAYTTSTCFKVVKTNKTYCLSIAEISNTLFGEFRIVPLLVEILKDNRA

SEQ ID NO: 20 - protein, 444 aa, Infectious bursal disease virus
QQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQAGGVTITLFSANIDAITSLSIGGELVFQTSVQGLILGATIYLIGFDGTAVITRAVAADNGLTAGTDNLMPFNIVIPTSEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRAIR

SEQ ID NO: 21 - protein, 1093 aa, Infectious bronchitis virus
MLVKSLFLVTILCALCSANLFDSDNNYVYYYQSAFRPPNGWHLQGGAYAVVNSTNYTNNAGSAHVCTVGVIKDVYNQSVASIAMTAPLQGMAWSKSQFCSAHCNFSEITVFVTHCYSSGSGSCPITGMIPRDHIRISAMKNGSLFYNLTVSVSKYPNFKSFQCVNNFTSVYLNGDLVFTSNKTTDVTSAGVYFKAGGPVNYSIMKEFKVLAYFVNGTAQDVILCDNSPKGLLACQYNTGNFSDGFYPFTNSTLVREKFIVYRESSVNTTLALTNFTFTNVSNAQPNSGGVNTFHLYQTQTAQSGYYNFNLSFLSQFVYKASDFMYGSYHPSCSFRPETINSGLWFNSLSVSLTYGPLQGGCKQSVFSGKATCCYAYSYKGPMACKGVYSGELSTNFECGLLVYVTKSDGSRIQTRTEPLVLTQYNYNNITLDKCVAYNIYGRVGQGFITNVTDSAANFSYLADGGLAILDTSGAIDVFVVQGIYGLNYYKVNPCEDVNQQFVVSGGNIVGILTSRNETGSEQVENQFYVKLTNSSHRRRRSIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLKPPSSPSGRSFIEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMNSLNKNFGAITSVIQDIYAPPDAIQADAQVDRLITGRLSSLSVLASAKQSEYIRVSQQRELATQKINECVKSQSNRYGFCGSGRHVLSIPQNAPNGIVFIHFTYTPESFVNVTAIVGFCVNPANASQYAIVPANGRGIFIQVNGTYYITARDMYMPRDITAGDIVTLTSCQANYVNVNKTVITTFVEDDDFDFDDELSKWWNDTKHQLPDFDDFNYTVPILNISGEIDYIQGVIQGLNDSLINLEELSIIKTYI

(19) United States
(12) Patent Application Publication
(10) Pub. No.: US 2025/0009868 A1
(43) Pub. Date: Jan. 9, 2025

(54) OVERCOMING ANTIBODY-INTERFERENCE IN AVIANS

(71) Applicant: Intervet Inc., Rahway, NJ (US)

(72) Inventors: Maria Cornelia Wilhelmina Van Hulten, Wageningen (NL); Munir Iqbal, Camberley (GB); Angita Shrestha, London (GB)

(73) Assignee: Intervet Inc., Rahway, NJ (US)

(21) Appl. No.: 18/576,496

(22) PCT Filed: Jul. 12, 2022
(86) PCT No.: PCT/EP2022/069513

§ 371 Date: Jan. 4, 2024

(30) Foreign Application Priority Data
Jul. 13, 2021 (EP) 21185334.6

(51) Int. Cl. A61K 39/145; A61K 39/00; A61K 39/12; A61K 39/17; A61K 39/215; A61K 39/395; A61P 37/04

(52) U.S. Cl. CPC A61K 39/145; A61K 39/12; A61K 39/17; A61K 39/215; A61K 39/3955; A61P 37/04; A61K 2039/552

(57) ABSTRACT

The present invention provides a recombinant protein, and a recombinant vector expressing that protein, that can be used for the vaccination of seropositive avians, whereby the antibodies in the avian target are specific for an antigen comprised in that recombinant protein. By comprising in the recombinant protein also a domain that can bind to a cell surface protein on avian antigen presenting cells (APCs), the antigen is targeted to those APCs. It was found that this type of vaccine could safely overcome the negative effects of antibody interference, even after a single dose, even in very young avians, and even in the context of very high antibody levels.

### CROSS-REFERENCE TO RELATED APPLICATIONS

This application is a U.S. National Phase entry under 35 U.S.C. § 371 of International Patent Application No. PCT/EP2022/069513, filed Jul. 12, 2022, which published as WO2023/285489 on Jan. 19, 2023, which claims priority to EP Application No. 21185334.6, filed Jul. 13, 2021; the content of each of which is hereby incorporated by reference in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14463-291-999_SEQ_LISTING.xml”, was created on Aug. 30, 2024, and is 30,030 bytes in size.

The present invention relates to the field of vaccination of avians; more specifically the invention relates to a recombinant protein for use in a method to protect an avian that possesses antibodies reactive with the antigen in said protein. In particular the invention relates to a recombinant protein, a recombinant vector, and a vaccine for use in said method. Further the invention relates to a use and a method for the treatment of avians by administration of the protein, the vector or the vaccine.
As a nutritious and affordable source of protein, avian meat and eggs are a prominent part of the diet of most of the world's human population. The main species of poultry bred for such economic purposes are chickens, turkeys, ducks and geese. To raise these birds in the large numbers that are required, while maintaining their health and well-being, the poultry industry is keen to optimise management conditions, and to provide good veterinary care. A vital part of this strategy is the prophylactic protection by vaccination against a wide variety of avian pathogens that may cause infection and disease, with sometimes devastating effects on animal well-being and the economy of operation. For many years a wide variety of vaccines have been commercially available against most of the viral-, bacterial-, and parasitic diseases that may affect avians of economic relevance. Such vaccines can be of different types, such as live attenuated, inactivated, subunit, nucleic acid, viral vector, etcetera.

Especially for the poultry that is produced in very large numbers, i.e. meat-type birds (broilers), it is common practice to protect young birds as early as possible. However the active vaccination of very young animals with an immature immune system is often not very successful. Therefore an effective work-around is by the vaccination of their mothers before and during their egg-laying period. The maternal antibodies generated by the hens are transferred to the egg with the yolk, which is internalised by the developing chick. This way the chicks can be passively protected by these maternally derived antibodies (MDAs) against a variety of pathogens, already at their day of hatch. However most of the MDAs have worn off again in 3 weeks' time due to biological degradation, therefore an active vaccination of the growing chicks themselves must also be provided to induce a proper immune-protection after the first weeks. At that time some MDAs may still be present in the birds.

A similar situation of vaccination in the context of pre-existing antibodies, arises in the case of birds of older age which have antibodies induced by a prior vaccination that slowly wear off, so that a booster vaccination is required, in order to restore antibody titres to protective levels.

An important veterinary and scientific puzzle arises in deciding when a vaccination can be given to birds that already possess antibodies which are reactive with an antigen that is comprised in the vaccine to be used. Clearly, vaccinating when antibody titres have almost gone is too late as that leaves a gap period between the drop of those antibody titres below protective level, and the onset of protection from the active immunisation. In this gap period the birds are vulnerable to infection and disease.

However, vaccinating when the birds still have considerably high titres of circulating antibodies, is too early, as that often affects the efficacy of the vaccination; this happens probably because those antibodies in some way bind to and sequester the vaccine antigens, which may speed up their degradation and/or prevent them from inducing a proper immune-response. This last phenomenon is called ‘antibody-interference’, a variant of which is where this regards MDA: ‘MDA-interference’. This is a well-known problem for the effective vaccination against the main pathogens that affect the poultry industry worldwide. Examples of these main pathogens are: infectious bursal disease virus (IBDV; a.k.a. Gumboro disease virus), Infectious Bronchitis virus (IBV), Newcastle disease virus (NDV), and Avian Influenza virus (AIV; a.k.a. fowl plague virus); the last two are even notifiable diseases of the OIE [World Organisation for Animal Health].

For these diseases antibody-interference is well-known to diminish vaccination efficacy which leaves the birds vulnerable to field infection, especially when they are kept in close proximity, and/or in areas with a high prevalence of an avian pathogen.

Over time, many different approaches have been tried to overcome antibody-interference, in order to prevent a gap in protection, and to optimise the vaccination of seropositive avians. The more straightforward attempts for overcoming antibody-interference included adaptations to the vaccine to increase antigen dose, and/or to use (stronger) adjuvants. Also more virulent, c.q. less attenuated, strains of live vaccine-pathogens have been tried in a hope those could break-through higher titres of antibodies and thus could be administered at an earlier time. As these methods are not generally satisfactory, more complicated approaches were tested.

For the active vaccination of young birds against IBDV, one method involves the monitoring of their MDA levels by serological testing of a sample of the birds to determine the optimal date for the vaccination. However the result is that effective active vaccination can only be applied at 2-3 weeks of age, and a protection gap for many of the birds is inevitable because of the variations in a large flock. Alternatively, ‘complexed IBDV vaccines’ (live attenuated vaccine-virus bound by antibodies) have been administered at early age, whereby the antigen is only released at a later time. Also viral vector systems have been used, for example using a fowl-pox-, or an avian herpes virus, as a vector for expressing the main IBDV antigen, the viral protein 2 (VP2). This was reviewed by Müller et al. (2012, Avian Pathol., vol. 41, p. 133-139).

For NDV, different approaches in vaccination have been applied, but antibody-interference is still a problem today; for a review see: Dimitrov et al. (2017, Vet. Microbiol., vol. 206, p. 126-136).

Even the use of recombinant vector vaccines may suffer from antibody-interference, for example in the case the antibodies react with the vector virus itself and/or with the antigen it expresses, see: Hu et al. (2020, Vaccines, vol. 14, p. 222, doi: 10.3390). For NDV as vector, solutions considered were e.g. to change the serological profile of the NDV vector (Steglich et al., 2013, PLOS One, vol. 8, e72530), or to select a strain of NDV that allegedly is less inhibited by anti-NDV antibodies (EP 2998315).

For IBV, MDA are well-known to interfere with vaccination of 1 day old chicks, see: Terregino et al., 2008 (Avian Pathol., vol. 37, p. 487-493).

For AIV, the relevance of effective vaccination even extends beyond the veterinary field, as this virus can give rise to zoonotic infections of humans with pandemic potential. Over time many different approaches of using classical- or recombinant AIV vaccines have been tried, with varying levels of success, see: D. Swayne, 2009 (Comp. Imm. Microbiol. and Inf. Dis., vol. 32, p. 351-363). However, and similar to the situation for several other vaccines, dealing with the interference by AIV-reactive antibodies still remains to be a problem (Murr et al., 2020, Avian Dis., vol. 64, p. 427-436).

Consequently, in spite of the many different approaches tested in the field of avian vaccination, there still is a pressing need for an effective way to overcome the negative effect that pre-existing antibodies in the target animals have on the efficacy of vaccination with an antigen to which these antibodies can bind.
Shrestha et al. (2018, Vaccines, vol. 6, p. 75, doi:10.3390) reviewed options for improving the vaccination of avian targets, by the selective targeting of an antigen to antigen-presenting cells (APCs). A wide variety of ways are described to achieve such targeting, e.g.: by using ligands, antibodies, nanoparticles, viral vectors, or cell-penetrating peptides. No method for overcoming antibody-interference in avians is described or suggested.

WO 2017/055235 describes antigen-targeting to antigen-presenting cells (APCs), but employs antigen-internalisation. The treatments described are exclusively for mammalians, specifically cats and dogs, and are aimed at the reduction of allergies. Antibody interference is not discussed.

Jauregui et al. (2017, Res. Vet. Sci., vol. 111, p. 55-62) describe the targeting of AIV HA antigen to dendritic cells in chickens. Purified H5 HA antigen was chemically conjugated to a mouse monoclonal antibody directed against one domain of Dec-205. This conjugate was used to vaccinate chickens of 21 weeks of age. As all chickens employed were seronegative for anti-HA antibodies (see Jauregui, FIG. 7, day 0), thus Jaurequi et al., do not describe or suggest overcoming antibody interference in seropositive avians.

It is therefore an object of the present invention to overcome one or more disadvantages in the prior art, by providing an effective way of overcoming the negative effect of antibody-interference on the vaccination of avian targets.
Surprisingly it was found that this object can be met, and consequently one or more disadvantages of the prior art can be overcome, by providing a method for the protection of avians that possess antibodies reactive with an antigen that is comprised in the vaccine to be administered, namely by targeting the antigen to APCs of the avian.

In the experiments as described hereinafter in detail, chickens with high or medium antibody levels received either targeted or untargeted vaccine. The results show a spectacular difference in the vaccination efficacy, in favour of the targeted antigen. On the contrary, untargeted vaccine and a classic control vaccine hardly produced any response in the seropositive avians. Consequently, this method of protecting seropositive avians was able to effectively overcome the negative effect of antibody-interference on vaccination, and is unexpectedly efficacious, even with only a single dose, and even in very young birds.

Consequently, the inventors were properly surprised to find that this antigen-targeting, particularly in the context of pre-existing antibodies, on the one hand worked well even in immune-immature avians, and on the other hand did not give rise to any vaccine-enhanced disease, or vaccine-induced immune-disturbance, such as by overstimulation of the immune-system, auto-immunity, or induction of tolerance.

This method to protect an avian is equally applicable when not using the targeted antigen directly, but as a recombinant vector, for example a DNA plasmid, an RNA molecule, or a vector virus, that expresses the recombinant protein.

In addition, because this favourable effect is considered to be caused by the targeting and thus is independent of the antigen that is employed, it is perfectly conceivable that this method will also be equally successful using a different antigen. This method thus enables the protection against a variety of avian pathogens for which the vaccination normally suffers from antibody-interference, such as e.g. NDV, IBDV, AIV and others.

It is not known exactly how or why this method of vaccination can break-through high antibody levels and still induce such an effective protective immune response. Although the inventors do not want to be bound by any theory or model that might explain these findings, they speculate that it is the targeting of the antigen to the APC, which in some way reduces clearing of the antigen by the pre-existing antibodies against it.
The success of the use of antigen targeting to APCs for the vaccination of avians with pre-existing antibodies against the vaccine antigen, was in no way predictable from any prior publication. This mainly because the mechanism of how antibody-interference works (blocking, masking, crosslinking, neutralising etc. of the vaccine antigen) is still not well understood today. This is especially true for antibody-interference in avians as that is a poorly studied animal system.

Further, while the first studies on antigen targeting were described already in the 1980's, these were aimed at human cancer therapy. Later a more general use in (predominantly human-) vaccination was considered. This is reviewed by Keler et al. (2007, Oncogene, vol. 26, p. 3758-3767).

Also, in some instances (maternal) antibodies have been responsible for enhancing viral diseases by antibody-dependent enhancement, this is called: vaccine-enhanced disease. This effect has been observed for a variety of viruses such as Lentiviruses and Dengue virus (Huisman et al., 2009, Vaccine, vol. 27, p. 505-512), and most recently for SARS-CoV-2 (Lee et al., 2020, Nat. Microbiol., vol. 5, p. 1185-1191). It was therefore a genuine concern that targeted vaccination could evoke such unwanted effects upon subsequent contact with the corresponding pathogen.

In addition, a translation from the mammalian- to the avian situation is far from straightforward as there is only little information on the functioning of the avian immune system, as compared to that in mammals/humans. Also the general review of Shrestha et al. (supra) does not enable particular methods, or take away any of the hesitations a skilled person would have in employing antigen-targeting. This because it could be feared to give rise to a type of immune-disturbance, and/or require a matured immune system.

Combined, this lack of information, and potential for complications made the use of antigen targeting to APCs an unlikely option for the vaccination of avians that have high levels of circulating antibodies reactive with the antigen in the vaccine. In addition, the choice of this method of vaccination for young avians was particularly uncertain, as the immune system of an avian at hatch is not yet matured so that it was unpredictable whether its APCs did already display suitable target proteins at their surface, and were matured enough to be able to transfer the binding to such surface protein into a productive stimulation of the animals' immune-system.
Therefore in one aspect the invention relates to a recombinant protein comprising an antigen and a binding domain that is capable of binding to a cell surface protein on an avian antigen-presenting cell (APC), for use in a method to protect an avian that possesses antibodies reactive with said antigen, against a pathogen from which said antigen was derived.
A “recombinant protein” is a protein of which the amino acid sequence is man-made and artificial. For the invention the recombinant protein can be obtained via molecular cloning- and recombinant protein expression techniques. After expression the protein can be isolated from the expression system, processed and purified when desired, and can subsequently be formulated into a composition suitable for use in the method to protect of the invention. Alternatively, the recombinant protein can be expressed and delivered via a recombinant vector, e.g. a DNA plasmid, an RNA molecule, or a viral vector, as described below.

Such techniques are well-known in the art and are disclosed in great detail in standard text-books like Sambrook & Russell: “Molecular cloning: a laboratory manual” (2001, Cold Spring Harbour Laboratory Press; ISBN: 0879695773); and: Ausubel et al., in: Current Protocols in Molecular Biology (J. Wiley and Sons Inc, NY, 2003, ISBN: 047150338X).

For the invention, the term “protein” incorporates similar terms such ‘peptides’, ‘oligopeptides’ and ‘polypeptides’.

The recombinant protein for use according to the invention is a fusion protein, composed of polypeptides from different origins, such as the antigen and a binding domain, both as defined for the invention, and optionally one or more peptides such as linkers, markers, etcetera, all connected in one amino acid chain.

The term “comprising” (as well as variations such as “comprises”, “comprise”, and “comprised”) as used herein, intends to refer to all elements, and in any possible combination conceivable for the invention, that are covered by or included in the text section, paragraph, claim, etc., in which this term is used, even if such elements or combinations are not explicitly recited; and not to the exclusion of any of such element(s) or combinations.

Therefore any such text section, paragraph, claim, etc., can therefore also relate to one or more embodiment(s) wherein the term “comprising” (or its variants) is replaced by terms such as “consisting of”, “consists of”, or “consist essentially of”.

An “antigen” is commonly known as a molecule that can interact with elements of the immune system such as antibodies and lymphocytes, which interaction may give rise to a humoral- and/or cellular immune response.

The sections of the antigen that are recognised by the immune system are called ‘epitopes’, which can be of linear or three-dimensional type. A 3D epitope is typically formed by the folding of a larger protein. A linear epitope needs to be of sufficient size e.g. at least 5 amino acids, either on its own or by being connected to a carrier molecule, e.g. by being comprised in a recombinant protein for use according to the invention.

The antigen is a polypeptide, thus: an antigenic polypeptide, contains at least one epitope, and is “derived” from a pathogen. For the invention ‘derived’ refers to the way the coding sequence for a particular antigen is selected, typically by analysis of the genetic information of the pathogen and its protein repertoire. The selected sequence is then recombined into a construct encoding the recombinant protein for use according to the invention.

For the invention, the antigen selected may thus be the whole or a part of a protein from a pathogen, wherein the pathogen is selected from a virus, a bacterium, a parasite, and a fungus.

The antigen can be derived from the natural sequence of an antigen from a pathogen, or can be an assembly, for example: have an amino acid sequence that is a consensus from several homologues of the antigen to be expressed, such as e.g. the same type of protein but derived from a variant of the pathogen, such as a different species, serotype, subtype, strain, isolate, etcetera. As is well-known, to obtain such a consensus sequence, either amino acid- or encoding nucleotide sequences can be compared and a consensus sequence can be derived from that comparison; for example by aligning several H9 HA nucleotide sequences using an appropriate computer program.

The antigen for the invention can also be a chimeric antigen, consisting of assembled parts from different antigens, biologically related or not. Further the sequence encoding the antigen can be subjected to ‘codon optimisation’, as is described below.

For the invention the antigen is selected from proteins that can generate a protective immune response against the pathogen from which the antigen was derived. For example, selected from: the VP2 protein from IBDV; the fusion (F)- or the hemagglutinin-neuraminidase (HN) protein of NDV; the spike protein from infectious bronchitis virus (IBV); and the HA- or the neuraminidase (NA) protein of AIV.
A “binding domain” for the invention is derived from the antigen-binding site of an immunoglobulin molecule and can be a part of an antibody comprising one or more of the complementarity-determining regions, for example can be a ‘single chain variable fragment’ (scFv) polypeptide.
For the invention, the binding domain “is capable of binding”. This refers to a binding that is specific, i.e. with sufficient avidity, to differ from any non-specific- or background binding. The difference between specific- and non-specific binding is well-known to the skilled artisan and can readily be distinguished for example in an in vitro binding assay, by diluting-out either the binding domain or the ligand; any non-specific binding is typically lost rapidly, e.g. at 1:10 or 1:100 dilution, while specific binding remains even with higher dilutions.
An “APC” is well known to be a cell of the lymphoid system that is capable of processing antigenic molecules and presenting (parts of) those molecules to the immune system of a human or animal. This presentation induces a cascade of reactions leading to the immune-maturation and -stimulation that is at the basis of a protective immune-response. APCs are e.g. B-lymphocytes, dendritic cells, macrophages, and natural killer cells.

A “cell surface protein on an avian APC” is a protein that is attached to- or anchored in the external side of the cell-membrane of an APC. These proteins play a role in the APC's functions in detecting and signalling. Many of the cell-surface proteins on APCs are members of the immunoglobulin superfamily of proteins. Examples of APC surface protein are e.g. CD83 and CD11c proteins. The ‘CD’ notation refers to ‘cluster of differentiation’, which is an international protocol for the classification and identification of surface proteins on cells of the lymphoid system.

An “avian” for the invention is any animal of the taxonomic Class Aves that is of economic- or of (veterinary) medical relevance. For example: chicken, turkey, duck, goose, quail, guinea fowl, partridge, pheasant, pigeon, falcon, and ostrich.
The terms “for use in a method to protect an avian” refer to the medical use of the recombinant protein for use according to the invention and as defined herein. The use can be of the protein directly or can be of the protein indirectly via expression from a recombinant vector.

For the invention the “method” applied refers to vaccination.

The term “protect” refers to the effect of the method for the invention, namely to a protective immune response that is induced by the method, namely by vaccination. Such an immune response protects the vaccinated avian against infection and/or disease caused by the pathogen from which the antigen (present in the recombinant polypeptide for use according to the invention) was derived.

The method to protect regards a reduction, in whole or in part, of the establishment or the proliferation of a productive infection by the pathogen in cells and organs of a susceptible avian, or of the subsequent signs of disease. This is achieved for example by reducing the pathogen's load or shortening the duration of the pathogen's replication. In turn this leads to a reduction in the avian of the number, the intensity, or the severity of lesions and associated clinical signs of disease, that can be caused by the infection with the pathogen.

Such reduction of infection or disease can readily be detected, for instance by monitoring the immunological response following vaccination with the recombinant protein for use according to the invention, and by testing the appearance of clinical symptoms or mortality after a (challenge) infection of vaccinated avians, e.g. by monitoring the avian' s signs of disease, clinical scores, serological parameters, or by re-isolation of the infecting pathogen. These results can be compared to a response to a similar infection in mock-vaccinated avians. Several ways to assess infection and symptoms of disease for the main avian pathogens are well-known in the art.

The protection against infection or disease by the method for the invention, provides immunised avians with an improvement of health, welfare, and economic performance. This can for instance be assessed from parameters such as an increase of well-being, survival, growth rate, feed conversion, and production of eggs, as well as reduced costs for (veterinary) health care.
The avian to be protected by the method of the invention, “possesses antibodies”. This applies to the moment in time when the method of the invention is applied: the time of vaccination. Whether an avian indeed has such antibodies can readily be determined e.g. by taking a blood sample from the avian around the time of vaccination and determining the titre of the antibodies against the antigen using standard serological methods. This does however not require that the determination itself of the value of that pre-existing titre, i.e. the performance of the serological test on a serum sample taken around the time of vaccination and/or the analysis and interpretation of the results of that test, is done at that time. Similarly, this does not prevent that the pre-existing titre at the time of vaccination is calculated and extrapolated from the level determined in a sample taken some time before the vaccination.
For the invention an avian “possesses” antibodies against an antigen when the titre of the antibodies reactive with that antigen in serum from that avian is above a background level. Such a background level is typically the level as present in a comparable avian that is naïve for the antigen or pathogen of interest. For the invention, this background level can conveniently be taken e.g. from the titre present in the serum of an SPF (specific pathogen free) avian of the same age and species.
The pre-existing antibodies can result from a passive transfer, as is typically the case with antibodies that were obtained from the mother via the egg-yolk. Such a seropositive avian would be called ‘MDA positive’, or ‘MDA+’. This applies to avians of very young age, e.g. from day of hatch (i.e. 1 day old), to about 3 weeks of age. Alternatively, the pre-existing antibodies can result from an active immunisation that the avian to be protected received earlier, and which resulted in the generation of antibodies; this applies to avians from about 3 weeks of age.
The terms “reactive with”, or its synonym: ‘specific for’, describe the capability of the pre-existing antibodies to interact with the antigen comprised in the recombinant polypeptide for use according to the invention, by a specific immune recognition. Similar terms are also ‘can bind to’, ‘can recognise’, etcetera, for as far as those refer to specific binding.

The unexpected advantageous effect of the present invention is prominent in the case that the pre-existing antibodies (in the avian to be protected) are reactive with the antigen that is comprised in the recombinant protein of the invention. In that situation an antibody-interference would normally occur which would reduce the efficacy of the protection.

The terms “a pathogen from which said antigen was derived” serve to indicate that the pathogen against which the method for the invention intends to protect, contains the antigen as defined above. This includes also homologues of the antigen and/or variants of the pathogen.

As the skilled person will understand, the match between the antigen in the recombinant protein for use according to the invention, and the pathogen against which the avian is to be protected, forms the basis for the protective immune response induced.

Details of embodiments and of further aspects of the invention will be described below.
In an embodiment of the recombinant protein for use according to the invention, the avian APC is selected from: a B-lymphocyte, a dendritic cell, a macrophage, and a natural killer cell.

Each of these cell-types can clearly be distinguished using standard serological- and biochemical methods, for example using determination based on proteins with CD designation, as described below.

In a preferred embodiment of the recombinant protein for use according to the invention, the avian APC is a dendritic cell.

In an embodiment of the recombinant protein for use according to the invention, the cell surface protein on the avian APC is selected from: Cluster of differentiation 83 (CD83), Cluster of differentiation 11c (CD11c), and dendritic cell receptor for endocytosis-205 (Dec205).

All these proteins are well-known in this field and are surface proteins on APCs: CD11c is a transmembrane protein on dendritic cells and some other APCs, which plays a role in the activation of neutrophils. A CD11c-specific scFv comprises the amino acid sequence of SEQ ID NO: 18.

Dec-205 is an endocytic receptor on dendritic cells and lymphocytes. An example of a chicken Dec-205 is presented in GenBank accession number: AJ574899. A Dec-205-specific scFv comprises the amino acid sequence of SEQ ID NO: 19.

CD83 is a surface glycoprotein which belongs to the immunoglobulin superfamily. It is predominantly expressed on dendritic cells, and to a lesser extent also on lymphocytes and macrophages. It is a well-known marker for mature dendritic cells. An example of an avian CD83 is the protein presented in GenBank accession number XP_040519591.

In a preferred embodiment of the recombinant protein for use according to the invention, the cell surface protein is CD83.
In an embodiment of the recombinant protein for use according to the invention, the binding domain comprises the antigen binding site of an antibody.

In a preferred embodiment of the recombinant protein for use according to the invention the binding domain is a single-chain variable fragment (scFv).

As is well-known, an scFv is the smallest part of an immunoglobulin which retains one complete antigen binding domain but lacks the Fc part. An scFv is a single peptide which is itself a fusion construct, comprising one variable light chain (vL), a linker, and one variable heavy chain (vH). The order of these elements can be vL-linker-vH, or vH-linker-vL. In both cases the variable chains are oriented (relative to each other) as tail-to-head, whereby the c-terminal side is the tail.

In a preferred embodiment the order of the elements in the scFv is vH-linker-vL.

The linker sequence of an scFv provides a flexible region so that the two variable chains can orient themselves to form an antigen binding domain. In a preferred embodiment the linker sequence of the scFv comprises Glycine, and Serine or Threonine amino acids, and is from 10 to 50 amino acids long. In a more preferred embodiment the linker sequence of the scFv comprises the amino acid sequence (Gly₄-Ser)₄, as presented in SEQ ID NO: 1.

The specificities of the two variable chains of an scFv can be both for the same- or each for a different antigen. In a preferred embodiment, the two variable chains have the same specificity.

In an embodiment, the scFv is specific for CD83, in other words: is a CD83-scFv. Preferably, the scFv is specific for CD83 on an avian dendritic cell; more preferably the scFv comprises the amino acid sequence of SEQ ID NO: 2.

In embodiments of the binding domain, the scFv can be present two or more times.

In an embodiment of the recombinant protein for use according to the invention, the pathogen is pathogenic to avians. More preferably the pathogen is a virus. Even more preferably the virus is an RNA virus. Still more preferably the RNA virus is selected from: IBDV, NDV, IBV, and AIV. Still even more preferably the pathogen is selected from: IBDV, NDV, and AIV. Most preferably the pathogen is AIV.
In an embodiment of the recombinant protein for use according to the invention, the antigen is selected from: IBDV VP2 protein, NDV F protein, NDV HN protein, IBV spike protein, AIV HA protein, and AIV NA protein. More preferably the antigen is selected from one of AIV HA protein and AIV NA protein. Even more preferably the antigen is an AIV HA protein. Still more preferably the antigen is selected from an AIV HA protein of H5, H7 or H9 type.

All these viral protein antigens are well-known in this field, and many versions of their encoding sequences are readily available digitally in public sequence databases such as NCBI's GenBank and EMBL's EBI. Examples are: AIV H9 HA: GenBank acc.nr. ACP50708.1; NDV F: GenBank acc.nr. AAK55550.1; NDV HN: GenBank acc.nr. MH614933.1; IBDV VP2: GenBank acc.nr. KX827589.1; and IBV spike: GenBank acc.nr. AAA66578.1.

In addition, detailed information on HA proteins is available in the Research Collaboratory for Structural Bioinformatics (RCSB) Protein Data Bank (PDB) at: www.rcsb.org, and in the Influenza Research Database at: www.fludb.org.

In an embodiment of the recombinant protein for use according to the invention wherein the antigen is selected from an AIV HA protein, the antigen contains only the ectodomain of the HA protein. This can prevent attachment to the cell-membrane of cells used for the expression of the recombinant protein for use according to the invention.

The ectodomain of a mature AIV HA protein comprises the N-terminal part-without the signal sequence- and the central part of the HA protein, thus comprising the HA1 and HA2 domains, but not the transmembrane- and cytoplasmic domains; typically these last two sections together form the C-terminal 35-40 amino acids of an HA.

In an embodiment of the recombinant protein for use according to the invention wherein the antigen is the ectodomain of an AIV HA protein of the H5, H7, or H9 type, the antigen comprises a protein having the amino acid sequence selected from: SEQ ID NO's: 3, 4, and 5.

In an embodiment of the recombinant protein for use according to the invention wherein the antigen is an ectodomain from an AIV HA protein, the antigen also comprises a trimerization domain.

Such a trimerization domain can compensate for the loss of the transmembrane- and cytoplasmic domains of HA, and restore the ability to form a homo-trimer and resemble its natural 3D shape. Further it improves the solubility and stability of the recombinant protein of the invention with an HA-ectodomain antigen.

For the invention a trimerization domain is a peptide and can be one of several known to be suitable for this function, for example: the isoleucine zipper 3 domain of the GCN4 transcriptional activator from Saccharomyces cerevisiae, or the Foldon domain of the bacteriophage T4 fibritin protein (‘Foldon’).

In a preferred embodiment the trimerization domain is a Foldon; more preferably the Foldon comprises the amino acid sequence of SEQ ID NO: 6.

In an embodiment of the recombinant protein for use according to the invention wherein the antigen is an ectodomain of an AIV HA protein and the antigen also comprises a trimerization domain, the trimerization domain is situated at the C-terminal side (downstream) of the HA ectodomain.

In a preferred embodiment the HA ectodomain and the trimerization domain are placed in the recombinant protein for use according to the invention, without an intervening amino acid.

In a preferred embodiment, the antigen comprising the AIV H9 HA ectodomain and the Foldon, comprises the amino acid sequence of SEQ ID NO: 7.

In the recombinant protein for use according to the invention, the antigen and the binding domain can be placed in two orientations relative to each other, with either the antigen or the binding domain nearer to the N-terminal end of the recombinant protein for use according to the invention. In this regard, the trimerization domain that can be employed when the antigen is selected to be an HA ectodomain, is considered as part of the antigen.
In an embodiment of the recombinant protein for use according to the invention, the antigen is situated in said recombinant protein at the N-terminal side (upstream) of the binding domain.

In an alternate embodiment, the binding domain is situated in said recombinant protein at the N-terminal (upstream) side of the antigen.

In an embodiment, the recombinant protein for use according to the invention comprises a linker that is situated in-between the antigen and the binding domain, or in-between the binding domain and the antigen, depending on their mutual orientation. Preferably said linker is between 1 and 30 amino acids in size. More preferably the linker contains Glycine and Serine amino acids. Even more preferably said linker comprises the amino acid sequence of SEQ ID NO: 8.
Therefore, in an embodiment, the recombinant protein for use according to the invention comprises, one of the combinations selected from:
an AIV H5 HA ectodomain, a trimerization domain, a linker, and a CD83-scFv;
an AIV H7 HA ectodomain, a trimerization domain, a linker, and a CD83-scFv; and
an AIV H9 HA ectodomain, a trimerization domain, a linker, and a CD83-scFv;
   wherein the indicated elements are presented in N- to C-terminal direction.

In a preferred embodiment, the AIV HA ectodomain is selected from SEQ ID NO's: 3, 4, and 5; the trimerization domain is SEQ ID NO 6; the linker is SEQ ID NO: 8; and the CD83-scFv is SEQ ID NO: 2.

For the purposes of expressing, harvesting, quantifying, and (optionally) purifying the recombinant protein for use according to the invention, the recombinant protein may also comprise one or more peptides that function as a biochemical- or serological marker (or tag). The markers may be the same or different. The markers can be placed at different locations in the recombinant protein.

Well-known markers are: affinity tags such as a Maltose binding protein (MBP)- or Histidine (His)-tag; epitope tags such as Myc-, Ctag-, V5- or Flag-tag; or fluorescent protein tags such as a GFP or YFP, or a part thereof; all well-known in the art.

The marker can be used for detection and quantification purposes, e.g. for detection or binding with specific antibodies, e.g. in an IFT or an ELISA. Purification can be done e.g. using immune- or metal affinity chromatography.

A His-tag typically has from 4 to 10 histidines. Preferably the His-tag is a 6× histidine tag, i.e. has 6 consecutive histidines.

A “Ctag”, comprises SEQ ID NO: 9, and is the C-terminus of α-synuclein protein, which is known to cause aggregates found in neurological disorders such Parkinson's disease. When used, the Ctag is preferably comprised in the C-terminus of a recombinant protein for the invention. Ctag purification by immuno-affinity chromatography is sometimes more effective than His-tag purification, e.g. in case there is disturbance from protein in the culture of the expression system.

A V5 tag is derived from Simian virus 5. Preferably the V5 tag comprises the amino acid sequence of SEQ ID NO: 10.

In an embodiment, the recombinant protein for use according to the invention comprises a marker peptide. More preferably the marker peptide is one or more selected from a Ctag, a His tag and a V5 tag. Even more preferably the recombinant protein comprises 2 or more from a Ctag, a His tag and a V5 tag.
For the expression of the recombinant protein for use according to the invention some further adaptations can be made when desired. Such fine-tuning or optimisation is routine and is well-known to the skilled artisan. For example, depending on how the protein is to be expressed by host cells of an expression system: inside the cells, on their surface, or secreted to their exterior. In the last two cases a signal sequence can be provided at the N-terminal side, which signal functions well in the cells of the expression system to be used. An example is the use the ‘Drosophila melanogaster immunoglobulin heavy chain binding protein’ (BIP) signal sequence, to enable secretion when expressing in S2 cells.

In an embodiment the recombinant protein for use according to the invention comprises a signal sequence; preferably the signal sequence is a BIP signal sequence; more preferably the BIP signal sequence comprises the amino acid sequence of SEQ ID NO: 11.

In the course of the construction process of the nucleic acid that is to provide express of the recombinant protein for use according to the invention, one or more restriction enzyme (RE) sites may be used. When those RE sites are located in the coding region of the recombinant protein, their remaining nucleotides will translate into a few amino acids which are then located in-between some of the elements that make up the recombinant protein for use according to the invention.

For example, one construct used for the invention, employed RE sites Kpnl and Pacl to subclone the H9 HA ectodomain-Foldon element, and used RE sites Notl and Xbal to subclone the CD83-scFv at the C-terminal side of the HA antigen-Foldon and the linker of SEQ ID NO: 8.

As a result one version of a recombinant protein for use according to the invention comprises the amino acid sequence of SEQ ID NO: 12, the details of which are described in Table 1.

TABLE 1
Composition of SEQ ID NO: 12

| | Length of | Location of elements, |
|---|---|---|
| | elements | in aa nrs of SEQ ID |
| Elements | in aa | NO: 12 |
| Signal sequence: BIP | 18 | 1-18 |
| Kpnl RE site remains | 3 | 19-21 |
| Antigen: AIV H9 HA ectodomain | 491 | 22-512 |
| Antigen: Foldon trimerization | 29 | 513-541 |
| domain | | |
| Pacl RE site remains | 3 | 542-544 |
| Linker | 5 | 545-549 |
| Notl RE site remains | 3 | 550-552 |
| Binding domain: CD83-scFv | 248 | 553-800 |
| Xbal RE site remains | 2 | 801-802 |
| Marker: V5 tag | 14 | 803-816 |
| Marker: 6xHis tag | 6 | 817-822 |

A control construct, without the linker and the CD83-scFv was prepared. This construct lacked the region of SEQ ID NO: 12 from aa 545-802, and comprised the amino acid sequence of SEQ ID NO: 13.
Similar constructs to SEQ ID NOs: 12 and 13 can readily be made using one of the other HA antigen sequences: a H5 HA- or H7 HA ectodomain, for example as presented in SEQ ID NOs: 4 and 5 respectively.
In an embodiment of the recombinant protein for use according to the invention, the antibodies reactive with the antigen are maternally derived antibodies.

For the invention, it can readily be established whether the pre-existing antibodies are maternally derived or not: practically, only chicks of less than 2-4 weeks of age will have MDAs. Also, MDAs consists mainly of IgY, which is a functional homolog of mammalian IgG, but differs structurally: IgY has 4 heavy chain constant domains, compared to three in IgG.

In an embodiment of the recombinant protein for use according to the invention, the avian to be protected is a poultry. More preferably the poultry is selected from: chicken, turkey, duck, and goose. Even more preferred, the poultry is a chicken.
For the invention, the avian may be of any type, breed, or variety, such as: layers, breeders, broilers, combination breeds, or parental lines of any of such breeds. Preferred poultry types are selected from: broiler, breeder, and layer. More preferred are broiler- and layer type poultry. Most preferred are broiler poultry.
As described, the present invention provides a recombinant protein for use in a method to protect seropositive avians against pathogens. This method can advantageously be applied either in older birds where the pre-existing antibodies are the result of a prior active vaccination, or in young birds where the pre-existing antibodies are MDA.
Therefore, in an embodiment of the recombinant protein for use according to the invention, the avian to be protected is less than 4 weeks of age; preferably less than 3 weeks of age; more preferably less than 2 week of age; even more preferably less than 1 week of age, still even more preferably is 1 day old (i.e. at day of hatch). In an embodiment the avian to be protected is at about 18 days of embryonic development (i.e. in ovo).

In an alternate embodiment of the recombinant protein for use according to the invention, the avian to be protected is 2 weeks or more of age.

As described, the recombinant protein for use according to the invention can equally well be applied by an indirect use, namely by expressing the recombinant protein from a recombinant vector, e.g. a DNA plasmid, an RNA molecule, or a viral vector.
Therefore in a further aspect the invention relates to a recombinant vector capable of expressing the recombinant protein for use according to the invention, for use in a method to protect an avian that possesses antibodies reactive with an antigen that is comprised in the recombinant protein expressed by said recombinant vector, against a pathogen from which said antigen was derived.
A “vector” is well-known in the field of the invention as a molecular structure that carries genetic information (a nucleic acid sequence) for encoding a polypeptide, with appropriate signals to allow its expression under suitable conditions, such as in a host cell. For the invention ‘expression’ regards the well-known principle of the expression of protein from genetic information by way of transcription and/or translation.

Many types and variants of such a vector are known and can be used for the invention, ranging from nucleic acid molecules like DNA or RNA, to more complex structures such as virus-like particles and replicon particles, up to replicating recombinant micro-organisms such as a virus.

The recombinant vector for use according to the invention is a “recombinant”, as it has a molecular make-up that was changed by manipulation in vitro of its genetic information. The changes made can serve to provide for, to improve, or to adapt the replication, expression, manipulation, purification, stability and/or the immunological behaviour of the vector and/or of the protein it expresses. These, and other techniques are explained in great detail in standard text-books like Sambrook & Russell, and Ausubel et al., both supra, and: C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).
Depending on the type of vector employed more or less signals need to be provided for the replication and expression, either in cis (i.e. provided within the recombinant vector itself) or in trans (i.e. provided from a separate source), this is all well-known.

The skilled person is well equipped to select and combine the required signals into operational combinations to make the recombinant vector for use according to the invention “capable of expressing” the recombinant protein for use according to the invention under appropriate conditions. Next to elements to assist with the construction and cloning, such as restriction enzyme recognition sites or PCR primers, well-known elements can be selected from one or more of a: promoter, stop codon, termination signal, polyadenylation signal, 7-methylguanosine (7mG) cap structure, and an intron with functional splice donor- and -acceptor sites.

In embodiments of the recombinant vector for use according to the invention, the features of the recombinant protein, the use, the method, the protection, the avian, the antibodies, the antigen, and the pathogen, are all as embodied herein.
In an embodiment of the recombinant vector for use according to the invention, the recombinant protein it expresses comprises the amino acid sequence of SEQ ID NO: 12.
The nucleotide sequence used for the expression of the amino acid sequence of SEQ ID NO: 12, comprises the nucleotide sequence of SEQ ID NO: 14.

Therefore, in an embodiment of the recombinant vector for use according to the invention, the vector comprises the nucleotide sequence of SEQ ID NO: 14.

The control protein of SEQ ID NO: 13 is encoded by a nucleotide sequence comprising the nucleotide sequence of SEQ ID NO: 15.
Both SEQ ID NOS: 14 and 15 have been codon optimised towards the codon-usage table of D. melanogaster S2 cells, to optimise the expression in these cells. Details as described hereinbelow.
As described, the recombinant vector for use according to the invention can have several different forms.
Therefore in an embodiment, the recombinant vector for use according to the invention is selected from a nucleic acid, a virus, and a replicon particle (RP).
For the invention, the nucleic acid can be a DNA or an RNA, can be single or double stranded, and can be natural or synthetic in origin.
In an embodiment of the recombinant vector for use according to the invention wherein the vector is a nucleic acid, the nucleic acid is a eukaryotic expression plasmid.
A “eukaryotic expression plasmid”, usually of DNA, has the appropriate signals for expression of a heterologous gene that is inserted into the plasmid, under the operational control of a promoter that is active in a eukaryotic cell. The plasmid can then be inserted into a eukaryotic host cell or host organism by some method of transfection, e.g. using a biochemical substance as carrier, by mechanical means, or by electroporation, and will provide for the expression of the heterologous gene insert. Typically such expression will be transient, as the plasmid lacks signals for stable integration into the genome of a host cell; consequently such a plasmid will typically not transform or immortalise the host or the host cell. All these materials and procedures are well known in the art and are described in handbooks.

Such eukaryotic expression plasmids are commercially available from a variety of suppliers, for example the plasmid series: pcDNA™, pCR3.1™, pCMV™, pFRT™, pVAX1™, pCI™, Nanoplasmid™, pCAGGS etc.

In a preferred embodiment the eukaryotic expression plasmid is a pFRT plasmid (Thermo Fisher Scientific) or a pCAGGS plasmid (Niwa et al., 1991, Gene, vol. 108, p. 193-199).

A eukaryotic expression plasmid can comprise several features for regulation of expression, purification, etc. One possible signal is an antibiotic resistance gene, which can be used for selection during the construction and cloning process. However when intended for administration to a human or animal target, such antibiotic selection is not desired for fear of generating antibiotic resistance.

In a preferred embodiment of the recombinant vector for use according to the invention, wherein the vector is a nucleic acid, and the nucleic acid is a eukaryotic expression plasmid, the plasmid does not contain an antibiotic resistance gene.

The recombinant vector for use according to the invention, in the form of a eukaryotic expression plasmid, can be delivered to a host cell or target organisms, where it will express the HA stem polypeptide for the invention in the host cell. Delivery of the expression plasmid can be in several ways, e.g. by mechanical or chemical means, as naked DNA, or encapsulated with an appropriate (nanoparticulate) carrier, such as a protein, polysaccharide, lipid or a polymer. Well-known examples of nucleic acid carriers are dendrimers, lipid nanoparticles, cationic polymers and protamine.

A special form of the recombinant vector for use according to the invention, as a eukaryotic expression plasmid, is when the plasmid provides for the delivery of replicon RNA.
Therefore in an embodiment of the recombinant vector for use according to the invention, wherein the vector is a nucleic acid, and the nucleic acid is a eukaryotic expression plasmid, the plasmid encodes a replicon RNA.
A “replicon RNA”, is a self-replicating RNA which contains, in addition to the nucleic acid encoding the recombinant polypeptide for the invention, elements necessary for RNA replication, such as a replicase gene. However, unlike a replicon particle (RP), a replicon RNA is not packaged by viral structural proteins and is thus less efficient at entering host cells on its own.

The replicon RNA-encoding plasmid can be delivered to a host cell in the same way as a protein-expressing plasmid.

Vaccination with a eukaryotic expression plasmid encoding replicon RNA provides an advantage over vaccination with a eukaryotic expression plasmid expressing protein, because the replicon RNA provides for an amplification step: the translation of replicase makes the replicon RNA produce sub genomic messenger RNA encoding the recombinant protein for use according to the invention. This results in the expression of high amounts of the recombinant protein in the host cell, respectively in the target avian.

In a preferred embodiment of the recombinant vector for use according to the invention, wherein the vector is a nucleic acid, the nucleic acid is a eukaryotic expression plasmid, and the plasmid encodes a replicon RNA, the replicon RNA is an Alphavirus-based replicon RNA; more preferably the Alphavirus-based replicon RNA is a Venezuelan equine encephalitis virus (VEEV) based replicon RNA.
An example of a eukaryotic expression plasmid encoding a VEEV replicon RNA is e.g. a pVAX plasmid (Thermo Fisher Scientific), comprising VEEV non-structural protein genes 1-4, driven by a eukaryotic promoter such as a human CMV immediate early gene 1 promoter.
In an alternate embodiment of the recombinant vector for use according to the invention wherein the vector is a nucleic acid, the nucleic acid is an RNA molecule.

The RNA molecule for the invention can have different forms and functions, for example can be an mRNA or can be a replicon RNA.

A recombinant vector for use according to the invention, as an RNA molecule can be delivered to the avian or to a host cell in different ways, e.g. by mechanical or chemical means, or encapsulated with an appropriate (nanoparticulate) carrier, such as a protein, polysaccharide, lipid or a polymer, as described herein. To stabilise the RNA nucleotide-analogues can be incorporated, or certain chemical modifications may be applied, e.g. to the nucleotides or to their backbone.
In an embodiment of the recombinant vector for use according to the invention wherein the vector is a nucleic acid and the nucleic acid is an RNA molecule, the RNA molecule is an mRNA.
An “mRNA” (messenger RNA) is well-known in the art, and typically has a 5′ 7-methylGuanosine (7mG) cap and a 3′ poly-A tail. An mRNA can be delivered to a eukaryotic host organism or host cell by way of transfection and/or by using an appropriate carrier, e.g. a polymer or a cationic lipid.
In an embodiment of the recombinant vector for use according to the invention wherein the vector is a nucleic acid and the nucleic acid is an RNA molecule, the RNA molecule is a replicon RNA.
The replicon RNA can be produced in vitro e.g. using a pVAX plasmid as described herein, and then be administered to a host cell or a target organism, using any suitable method.
Recombinant vectors for the expression and delivery of a heterologous protein in the form of replicating recombinant virus vectors, are well-known in the art. These provide for an efficient method of vaccination, as the viral vector replicates and amplifies in the target avian. Assembly and modification of a recombinant vector virus is routine and can be done using standard molecular biological techniques.
Therefore in an embodiment of the recombinant vector for use according to the invention, the recombinant vector is a virus.
For the invention, the viral vector is a virus that replicates in an avian. Many different virus species have been used over time as recombinant vector for avians.
In an embodiment of the recombinant vector for use according to the invention wherein the vector is a virus, the virus is selected from a Herpesvirus, a Poxvirus, a Paramyxovirus, and an Adenovirus.
Examples of suitable vector viruses that can be used as vector for avians are well known in the art and are e.g. of a Herpesvirus: a Herpesvirus of Turkeys (HVT), or a Marek's disease virus (MDV) of serotype 1 or 2; of a poxvirus: fowl pox virus; of a Paramyxovirus: NDV; and of an Adenovirus: fowl Adenovirus.
In a preferred embodiment of the recombinant vector for use according to the invention wherein the vector is a virus, and the virus is a Herpesvirus, the Herpesvirus is selected from: HVT, MDV1 and MDV2.
Examples of recombinant viral vectors expressing and delivering an Influenza HA gene are described: for HVT as vector in WO 2012/052384 and EP19218804.3. For NDV as vector, an example is described in: WO 2007/106882.
For the construction of a recombinant viral vector, typically an expression cassette is inserted into a locus in the vector's genome. Different techniques are available to control the locus and the orientation of that insertion. For example by using the appropriate flanking sections from the genome of the vector to direct the integration of the cassette by a homologous recombination process, e.g. by using overlapping Cosmids as described in U.S. Pat. No. 5,961,982. Alternatively the integration may be done by using the CRISPR/Cas technology.

An ‘expression cassette’ is a nucleic acid fragment comprising at least one heterologous gene and one promoter to drive the transcription of that gene, to enable the expression of the encoded protein. The termination of the transcription may be provided by sequences provided by the genomic insertion site of the cassette, or the expression cassette can itself comprise a termination signal, such as a transcription terminator. In such a cassette, both the promoter and the terminator need to be in close proximity to the gene of which they regulate the expression; this is termed being ‘operatively linked’, whereby no significant other sequences are present between them that would intervene with an effective start-, respectively termination of the transcription. As will be apparent to a skilled person, an expression cassette is a self-contained expression module, therefore the orientation of its reading direction relative to the vector virus genome is generally not critical.

Other than the use of a virus as a vector for use according to the invention, the recombinant vector for use according to the invention can also be delivered and expressed to an avian by way of a macro-molecular structure that resembles a virion. Examples are virus-like particle (VLPs), or replicon particles (RPs). Known as ‘single cycle’ infectious particles, these structures contain features necessary to infect a host cell, and express the heterologous gene it carries, however, they will typically not be capable of full viral replication, for lack of (relevant parts of) the viral genome from which they were constructed. This serves as a built-in safety feature,

“RPs” are well-known, and several RPs have been developed as a platform for the expression and delivery of a variety of proteins. Favourable basis for an RP is an Alphavirus, because of its broad host-range and rapid replication. Of course appropriate safety measures need to be taken to attenuate and control the infection of such RPs, as some Alphaviruses are highly pathogenic in their wildtype form. For a review, see: Kamrud et al. (2010, J. Gen. Virol., vol. 91, p. 1723-1727), and: Vander Veen, et al. (2012, Anim. Health Res. Rev., vol. 13, p. 1-9.).

Therefore in an embodiment of the recombinant vector for use according to the invention, the vector is an RP. Preferably the RP is an Alphavirus RP. More preferably the Alphavirus RP is a VEEV RP.
Preferred Alphavirus RPs are based on VEEV, which have been applied as recombinant vector vaccine for human, swine, poultry, and fish. Methods and tools to construct, test, and use VEEV-based Alphavirus RPs are well-known and available, see for example: Pushko et al. (1997, Virology, vol. 239, p. 389-401), and: WO 2019/110481. Preferred VEEV RP technology is the SirraVax℠ RNA Particle technology (Harris vaccine).

The RNA for an RP can conveniently be produced in vitro: a DNA plasmid is used to translate a gene into RNA, which is harvested and transfected into a host cell together with helper RNA encoding in trans the VEEV structural proteins.

As described, the recombinant vector for use according to the invention can advantageously be used to deliver and express the recombinant protein for use according to the invention to an avian, e.g. as a way to vaccinate that target. This involves at some stage the administration of that vector to an avian, for example in the case the vector is a nucleic acid such as a DNA expression plasmid or an RNA molecule.

Also, the vector may be introduced into a host cell in vitro, for the amplification of the vector and/or the expression of the recombinant protein, after which the host cell (with the vector and/or the protein) is administered to the avian; for example in the case the vector is a viral vector, e.g. an HVT.

Still further, the vector may be introduced into a cell of a recombinant expression system for expression of the recombinant protein, and the protein be harvested from that cell culture, and used to vaccinate an avian as described above. Also, the host cell itself, infected or transfected with the recombinant vector for use according to the invention and containing and/or expressing the recombinant protein for use according to the invention, can be used for the method to protect for the invention, e.g. as the infected or transfected host cell may itself be used for the vaccination of an avian.

Depending on the type of vector applied, that introduction into a host cell may require a carrier, some method of transfection, or may be guided by the vector itself, as described herein.
Therefore in a further aspect the invention regards a host cell kept in vitro, said host cell comprising the recombinant protein for use according to the invention and/or the recombinant vector for use according to the invention.
A “host cell” for the invention, is a cell that allows the expression of the recombinant protein for use according to the invention, and/or allows the replication of the recombinant vector for use according to the invention.

A host cell for the invention can be a primary cell kept in vitro, and can be e.g. in a suspension, in a monolayer, or in a tissue.

Alternatively, the host cell can be an immortalised cell kept in vitro, for example a cell from an established cell-line, which can grow and divide almost indefinitely. Depending on the type of the host cell, the expression of the HA stem polypeptide for the invention will include more or less extensive post-translational processing, such as e.g. signal peptide cleavage, disulphide bond formation, glycosylation, and/or lipid modification.

The primary- and the immortalised host cell can be of the same- or from a different species. Also one or both can be of the same or of a different species as the avian that is the subject of the method to protect for the invention.

Much used host cells are fibroblasts and lymphocytes. In case of the use of HVT as recombinant vector virus for the invention, the host cells are preferably primary chicken embryo fibroblasts (CEF's), which can be used and stored as described, see e.g. WO 2019/121888.

In an embodiment of the host cell for the invention, said host cell is preferably an immortalised avian cell. Several immortalised avian cell-lines have been described, for example in WO 97/044443 and WO 98/006824; more preferably the immortalised avian host cell for the invention is an immortalised CEF; even more preferably an immortalised CEF as disclosed in WO 2016/087560.
In an embodiment of the host cell for the invention, said host cell is preferably a cell of a recombinant expression system. Examples of cells from expression systems are e.g. cells from bacteria, yeasts, insects, avians, or mammalians
Cells from bacterial expression systems are e.g. cells from the genera Escherichia, Bacillus, Salmonella, Caulobacter, or Lactobacillus.
Cells from yeast expression systems are e.g. cells from Saccharomyces cerevisiae or Pichia pastores.
Cells from insect cell expression systems are e.g. cells from Drosophila melanogaster, e.g. Schneider 2 (S2) cells, or cells for use in the Baculovirus-insect cell expression system: from Spodoptera frugiperda, e.g. Sf21 or Sf9 cells; or from Trichoplusia ni, e.g. High Five™ cells.
Cells from mammalian expression system are e.g. cells from hamsters, e.g. Chinese hamster ovary (CHO) cells.
   All these cell-lines and their corresponding use in a recombinant expression system are well known in the art and can be employed using routine techniques and materials.
   In an embodiment of the recombinant vector for use according to the invention, the nucleic acid that encodes the recombinant protein for use according to the invention, is codon optimised.
   Codon optimisation is well-known and is applied to improve the expression level of a gene in an expression system, which is typically a context that differs from that of the origin of the gene. The optimization involves the adaptation of the nucleotide sequence to encode the intended amino acids, but in a way that corresponds to the codon preference (the tRNA repertoire) of the recombinant vector, of the host cell, or of the target organism in which the sequence will be expressed. Consequently, the nucleotide mutations applied are silent.
   Therefore in an embodiment of the recombinant vector for use according to the invention, the recombinant protein for use according to the invention is encoded by a nucleic acid sequence that is codon optimised towards the avian organism that is intended to be protected by the method to protect for the invention. Preferably the codon optimisation is towards a poultry. More preferably the codon optimisation is towards a poultry selected from: a chicken, a turkey, a duck, and a goose.
   In an embodiment of the recombinant vector for use according to the invention, the recombinant protein for use according to the invention is encoded by a nucleic acid sequence that is codon optimised towards a cell of a recombinant expression system, preferably towards a cell from a bacterium, yeast, insect, avian, or mammalian. More preferably the nucleic acid is optimised towards an insect cell; even more preferably towards a Drosophila Schneider 2 (S2) cell.
   The recombinant protein for use and the recombinant vector for use, both according to the invention, can also be characterised by other wording to suit specific jurisdictions.
   Therefore, in a further aspect the invention regards the use of the recombinant protein for use according to the invention, or of the recombinant vector for use according to the invention, for the manufacture of a vaccine to protect an avian against a pathogen, whereby the antigen that is comprised in said recombinant protein or that is comprised in the recombinant protein expressed by said recombinant vector, was derived from said pathogen, characterised in that said avian possess antibodies reactive with said antigen.
   In embodiments of the recombinant protein, or of the recombinant vector, both for the manufacture of a vaccine according to the invention, the features of the recombinant protein, the recombinant vector, the protection, the avian, the pathogen, the antigen, and the antibodies, are all as embodied herein.
   A “vaccine” is well-known to be a composition comprising at least one compound that can induce a protective immunological effect, in a pharmaceutically acceptable carrier. The ‘immunologically active compound’ for the present invention is the recombinant protein for use, or the recombinant vector for use, both according to the invention.
   The manufacture of a vaccine for the invention can be done using routine methods and procedures all well known in the art. General techniques and considerations that apply to the manufacture of vaccines under well-known standards for pharmaceutical production are described for instance in governmental directives and regulations (Pharmacopoeia, 9CFR) and in well-known handbooks like “Veterinary vaccinology” and: “Remington” (both supra). Commonly such vaccines are prepared sterile and are prepared using excipients of pharmaceutical quality grade.

Such a manufacture will incorporate microbiological tests for sterility, and absence of extraneous agents, and may include studies in vivo or in vitro for confirming efficacy and safety. After completion of the testing for quality, quantity, sterility, safety and efficacy, the vaccine can be released for sale. All these are well-known to a skilled person.

For example when the recombinant protein for use according to the invention is produced by way of a recombinant expression system, the protein can be harvested from the expression system culture, e.g. as a whole culture. Alternatively the harvest can be as a part of such culture, e.g. the supernatant or the cell-pellet after centrifugation of the cell culture, or a filtrate or retentate after filtration. The superatant can be obtained after gravity settling of the culture, e.g. by standing overnight or by centrifugation; the filtrate is what passes through the filter upon filtration.
As described, the recombinant protein, and the recombinant vector, both for use according to the invention, achieve their advantageous effect in protecting an avian, through a vaccine comprising said recombinant protein and/or said recombinant vector.
Therefore, in a further aspect the invention regards a vaccine comprising the recombinant protein for use according to the invention, or comprising the recombinant vector for use according to the invention, and a pharmaceutically acceptable carrier, for use in a method to protect an avian that possess antibodies reactive with the antigen that is comprised in said recombinant protein or that is comprised in the recombinant protein expressed by said recombinant vector, against a pathogen from which said antigen was derived.
In embodiment of the vaccine for use according to the invention, the features of the recombinant protein, the recombinant vector, the use, the method, the protection, the avian, the antibodies, the antigen, and the pathogen, are all as embodied herein.
A “pharmaceutically acceptable carrier” is well-known to aid in the stabilisation and the administration of a vaccine, while being relatively harmless and well-tolerated by the vaccinee. Such a carrier can for instance be water or a physiological salt solution. In a more complex form the carrier can e.g. be a buffer, which can comprise further additives, such as a stabiliser or a preservant. Details and examples are for instance described in well-known handbooks such as: “Remington: the science and practice of pharmacy” (2000, Lippincott, USA, ISBN: 683306472), and: “Veterinary vaccinology” (P. Pastoret et al. ed., 1997, Elsevier, Amsterdam, ISBN 0444819681).
When the vaccine according to the invention comprises a recombinant vector that is a replicating virus, then the pharmaceutically acceptable carrier is preferably a composition stabilising that virus, or the host cell in which that virus is contained. Examples are several viral vaccine diluents, and stabilisers for frozen or freeze-dried storage, typically comprising e.g. a sugar, an amino acid, a physiological buffer (e.g. saline, PBS, or 50 mM HEPES), and often a bulky compound such as an albumin, a polymer etc. For example, when the vaccine comprises a recombinant HVT vector, such a vaccine is typically marketed as a cell-associated product. In that case the pharmaceutically acceptable carrier is preferably a mixture of culture medium, about 10% serum, and about 6% DMSO. This carrier also provides for the stabilisation of the HVT-infected host cells during freezing and frozen storage. The serum can be any serum routinely used for cell culturing such as foetal- or new-born calf serum.

When the vaccine according to the invention comprises a recombinant vector for use according to the invention that is a nucleic acid or an RP, the pharmaceutically acceptable carrier can be a simple buffer, e.g. a phosphate buffer with 5% w/v sucrose.

Further, an additional carrier can be added to stabilise and/or deliver the recombinant vector for a use in the invention, e.g. to encapsulate the recombinant vector according to the invention that is a nucleic acid or an RP with an appropriate (nanoparticulate) carrier, such as a protein, polysaccharide, lipid or a polymer. Preferably the additional carrier for a recombinant vector according to the invention that is an RP, comprises a nanogel that is a biodegradable polyacrylic polymer as described in WO 2012/165953.

Evidently, the recombinant vector or the in vitro host cell comprising such a vector, both for the invention, can be employed herein alive (i.e. replicative), or dead (non-replicative, or inactivated). In turn, only a part of the recombinant vector or the host cell, both for the invention, can be used for example as a: pellet, supernatant, concentrate, dialysate, extract, sonicate, lysate or as a fraction of a composition, e.g. a culture, comprising the vector and/or the host cell. All this is well-known to the skilled person.
When the vaccine for use according to the invention comprises the recombinant protein for use according to the invention, the vaccine can comprise an adjuvant to stimulate the immune response induced.
Therefore, in an embodiment, the vaccine for use according to the invention comprises an adjuvant.
An “adjuvant” is a well-known vaccine ingredient that stimulates the immune response of a target in a non-specific manner. Many different adjuvants are known in the art. Examples of adjuvants are: complete- or incomplete Freund's adjuvant, vitamin E or alpha-tocopherol, non-ionic block polymers and polyamines such as dextran sulphate, Carbopol™, pyran, Saponin, such as: Quil A™, or Q-vac™. Saponin and vaccine components may be combined in an ISCOM™. Furthermore, peptides such as muramyl dipeptides, dimethylglycine, and tuftsin. Also, aluminium salts, such as aluminium-phosphate or an aluminium-hydroxide which is available for example as: Alhydrogel™ (Brenntag Biosector), Rehydragel™ (Reheis), and Rehsorptar™ (Armour Pharmaceutical).

A much-used adjuvant is an oil, e.g. a mineral oil such as a light (white) mineral (paraffin) oil; or a non-mineral oil such as: squalene; squalane; vegetable oils or derivatives thereof, e.g. ethyl-oleate. Also combination products such as ISA™ (Seppic), or DiluvacForte™ and Xsolve™ (both MSD Animal Health) can advantageously be used.

A handbook on adjuvants and their uses and effects is: “Vaccine adjuvants” (Methods in molecular medicine, vol. 42, D. O'Hagan ed., 2000, Humana press, NJ, ISBN: 0896037355).

The adjuvant can be comprised in the vaccine for use according to the invention, in several ways. When the adjuvant comprises an oil, the vaccine can be provided in aqueous form, and can be formulated as an emulsion with the oil, in different ways: as a water-in-oil (W/O), an oil-in-water (O/W), or as a double emulsion, either W/O/W or O/W/O.
An “emulsion” is a mixture of at least two immiscible liquids, whereby one is dispersed in another. Typically the droplets of the dispersed phase are very small, in the range of micrometres or less. Procedures and equipment for the preparation of an emulsion at any scale are well-known in the art. To stabilise an emulsion, one or more emulsifiers can be used.

An “emulsifier” is a molecule with amphiphilic properties, having both a hydrophobic- and a hydrophilic side. Many emulsifiers are known in the art with their various properties. Most are readily available commercially, and in several degrees of purity. Common emulsifiers for vaccines are sorbitan monooleate (Span® 80) and polyoxyethylene-sorbitan-monooleate (polysorbate 80, or Tween® 80).

A well-known way to characterise the properties of (mixtures of) emulsifiers is the HLB number (hydrophile-lipophile balance; Griffin, 1949, J. Soc. Cosm. Chem., vol. 1, p. 311-326). Typically an emulsifier or emulsifier mixture with HLB number below 10 favours W/O emulsions, while an emulsifier (mixture) with HLB number of 10-16 will favour O/W emulsions.

Also an emulsion-stabiliser can be added; examples are benzyl alcohol, and triethanolamine.

In a preferred embodiment of the vaccine for use according to the invention, wherein the vaccine comprises an adjuvant, the adjuvant comprises an oil. More preferably the oil comprises a mineral oil. Even more preferably the mineral oil comprises a light (or white) liquid paraffin oil.

Examples of light liquid paraffin oils are: Drakeol® 6VR (Penreco), Marcol® 52 (Exxon Mobile), and Klearol® (Sonneborn).

In a preferred embodiment of the vaccine for use according to the invention, wherein the vaccine comprises an adjuvant, and the adjuvant comprises an oil, the vaccine is formulated as a water-in-oil emulsion.
In other wordings and for specific jurisdictions, further aspects of the invention can be defined as follows:
In a further aspect the invention regards the use of the recombinant protein for use according to the invention, or of the recombinant vector for use according to the invention, or of the vaccine for use according to the invention, to protect an avian against a pathogen, whereby the antigen that is comprised in said recombinant protein or that is comprised in the recombinant protein expressed by said recombinant vector, was derived from said pathogen, characterised in that said avian possess antibodies reactive with said antigen.
In an embodiment of the use according to the invention, the use comprises the administration to an avian of the recombinant protein, the recombinant vector, or the vaccine, all for the invention.
In embodiments of the use according to the invention, the features of the recombinant protein, the recombinant vector, the vaccine, the use, the method, the protection, the avian, the pathogen, the antigen, and the antibodies, are all as embodied herein.
In a further aspect the invention regards a method for protecting an avian against a pathogen, the method comprising the step of administering to said avian the vaccine for use according to the invention, whereby the antigen that is comprised in said vaccine was derived from said pathogen, and whereby said avian possess antibodies reactive with said antigen.
A vaccine for use according to the invention is typically prepared in a form that is suitable for administration to an avian, and that matches with a desired route of application, and with the desired effect.

Depending on the route of application of the vaccine for use according to the invention, it may be necessary to adapt the vaccine's composition. This is well within the capabilities of a skilled person, and generally involves the fine-tuning of the efficacy or the safety of the vaccine. This can be done by adapting the vaccine dose, quantity, frequency, route, by using the vaccine in another form or formulation, or by adapting one of the excipients of the vaccine (e.g. a stabiliser or an adjuvant).

The vaccine according to the invention in principle can be given to an avian by different routes of administration, and at different points in their lifetime; specifically the vaccine can be administered to an avian of any age that possess antibodies reactive with the antigen in the recombinant protein for use according to the invention.

When the administration is to be administered as early as possible, it can be administered at the day of hatch (“day one”), or even in ovo, e.g. at about 18 days of embryonic development, all well-known in the art.

Equipment for automated injection of a vaccine into a fertilized egg at industrial scale, is available commercially. This provides the earliest possible protection, while minimising labour costs. Different in ovo inoculation routes are known, such as into the yolk sac, the embryo, or the allantoic fluid cavity; these can be optimised routinely, when required.

The vaccine for use according to the invention can be formulated as an injectable liquid, suitable for injection, either in ovo, or parenteral.

In an embodiment the vaccine for use according to the invention is formulated as a liquid selected from a: suspension, solution, dispersion, and emulsion.

In an embodiment, the vaccine for use according to the invention is administered by parenteral route. Preferably the parenteral route is by intramuscular- or subcutaneous route.
The exact amount of the recombinant protein or of the recombinant vector, both for the invention, is not critical and can readily be established by comparing the protective effects of different amounts.

Also, when the vaccine for use according to the invention comprises a viral vector, this can replicate in the vaccinated avian and only needs to be administered in an amount that is enough to establish a productive infection in the avian.

For example, when the viral vector for use according to the invention is a recombinant HVT, a suitable inoculum dose is between 1×10\hat{ }1 and 1×10\hat{ }5 plaque forming units (pfu) of the HVT for the invention per animal dose; preferably between 1×10\hat{ }2 and 1×10\hat{ }4 pfu/dose, even more preferably between 500 and 5000 pfu/dose; most preferably between about 1000 and about 3000 pfu/dose. Methods to count viral particles of the HVT for the invention are well-known.

When the HVT vector for use according to the invention is cell-associated, these amounts of the HVT are comprised in infected host cells.

The volume per animal dose of the vaccine for use according to the invention can be optimised according to the intended route of application: in ovo inoculation is commonly given in a volume of 0.01 to 0.5 ml/egg, and parenteral injection in an avian is commonly given in a volume of 0.1 to 1 ml/bird.

Determination of what is an immunologically effective amount of the vaccine according to the invention, or the optimisation of the vaccine's volume per animal dose, are both well within the capabilities of the skilled artisan.

The dosing regimen for applying the vaccine for use according to the invention to an avian can be in single or multiple doses, in a manner compatible with the formulation of the vaccine, and in such an amount as will be immunologically effective.

Preferably, the regimen for the administration of a vaccine for use according to the invention is integrated into existing vaccination schedules of other vaccines that the target avian may require, in order to reduce stress to the animals and to reduce labour costs. These other vaccines can be administered in a simultaneous, concurrent or sequential fashion, in a manner compatible with their registered use.

The invention is described herein in various aspects and embodiments. It should be understood that any combination of these is considered to be within the scope of the invention. However merely for conciseness, not every possible combination is outlined herein in full.
The invention will now be further described by the following, non-limiting, examples.

### EXAMPLES

### Example 1: Generating AIV-MDA Positive Chicken

### 1.1. Introduction

In order to be able to test vaccination of seropositive chicken, an animal model resembling the actual situation in the field was created. Specifically, AIV MDA positive offspring was generated, by repeated vaccination of parental hens intramuscularly, using an inactivated-adjuvated vaccine. Aim was to reach HI titres in the offspring that resembled those in the field: at least between 5 and 7 Log2.

### 1.2. Materials and Methods

SPF White Leghorn layer chickens were vaccinated to generate MDA positive hatchlings. All chickens were housed in isolation rooms with floor pens. All chickens were given food and water ad libitum for the duration of the experiment, and were kept under veterinary surveillance.

### 1.2.1. Preparation of Vaccine for MDA Generation:

An inactivated AIV vaccine was made by propagating an avian influenza A virus of H9N2 subtype in 10-day old embryonated SPF chicken eggs. Specifically this was AIV strain: A/Chicken/Pakistan-/UDL-01/2008 (‘UDL-01’), see: GenBank: ACP50708.1, and: Iqbal et al. (2009, PLOS One, vol. 4: e5788). At 72 hours post infection, eggs were refrigerated at 4° C., and virus was obtained by harvesting the allantoic fluid, which was cleared by centrifugation at 3.000 rpm for 20 minutes. Virus was titrated by plaque assay or TCID50 on Madin-Darby canine kidney (MDCK) cells.

The virus was inactivated chemically using 0.1% Beta-propiolactone, after which three blind passages were performed in 10 day old embryonated SPF chicken eggs, to confirm inactivation. The inactivated virus harvest was then concentrated by ultracentrifugation at 27.000 rpm for 2 hours at 4° C. Next the inactivated virus was adjuvated with a liquid light paraffin oil, and formulated into a water-in-oil emulsion. The resulting vaccine had a titre of 1040 haemagglutination units (HAU)/ml.

### 1.2.2. Vaccination of Hens and Generation of MDA+ Hatchlings

A group of 40 SPF White Leghorn layer hens of 17 weeks old, were used. The chickens were marked individually. They were immunised with 0.5 ml of the inactivated-adjuvated H9N2 virus vaccine, at 520 HAU/dose, which was administered i.m. in the leg. The first dose of vaccine was given at 17 weeks of age (T=0), followed by second and third doses at 20 weeks of age (T=3 weeks post first dose) and 41 weeks of age (T=24 weeks post first dose), respectively.

Blood samples were collected from the wing vein of the hens at day 0 and at weeks 5, 11, 18, 29 and 36 after the first dose, for serological monitoring of the developing anti-AIV HI titre. Five SPF roosters were included in the group for fertilization, but these were not part of the actual study.

Fertilized eggs were collected from 36 weeks after the first vaccination dose. These were set to incubate until hatch. 10 hatchlings were sacrificed at day old (D0) to determine their level of MDA. Their hatchmates were used in the MDA-vaccination experiment.

1.2.3. HI assay
For HI assays, International guidance was complied with (WHO 676 global influenza surveillance network: manual for the laboratory diagnosis and virological surveillance of influenza. 153 (2011)). In short: two-fold serial dilutions of the sera were prepared by mixing 25 μl of serum with 25 μl PBS. Next, 4 HA units of influenza virus was added to the diluted serum and incubated at 37° C. for 1 hour. Finally, 50 μl of 1% chicken red blood cells were added to the serum-virus mixture and incubated at room temperature for 45 minutes. HI titres were expressed as the reciprocal of the highest dilution of antiserum that caused a total inhibition of the 4 units of virus hemagglutination activity.

The virus used in the HI assays was AIV H9N2 of strain UDL-01.

### 1.3. Results

The results of the hyper-immunisation of the mother hens to generate AIV MDA+ offspring, are depicted in FIG. 1. HI titrations were done with the homologous UDL-01 strain.

As a drop in HI titres was observed in the hen's sera at 18 weeks post start, a third vaccination was given. This resulted in very high HI titres in the hens, which remained at that level until the last sampling point.

Fertilized eggs were collected at 36 weeks post start (53 weeks of age), when the average (n=10) HI titre of the hens was 4096 (12 Log2).

As is clear from these results, and indicated in FIG. 1, there were significant differences between the HI titres at T=11 and T=18 weeks (p<0.05), and between T=18 and T=29 (p<0.001).

The HI titres induced by the MDA in the (unvaccinated) offspring from these hens was measured, at day of hatch and over time: at day 1, 7, 14, 21, 28, 35, 42, 56, 70 and 84 post hatch. Results are depicted in FIG. 2. HI titrations were done with the homologous UDL-01 strain.

At day 1, the HI titres in the chicks averaged (n=10): 588 (9.2 Log2). This titre had dropped slightly (not significant) at day 7 of age, but was more than halved at day 14 of age, at 181 (7.5 Log2), and rapidly declined further after that: at day 35 the average (n=10) HI titre was 16 (4 Log2), and no HI titre was detectable anymore at day 42 of age.

The International standard for protection from AIV mortality as defined by the OIE (www.oie.int/fileadmin/Home/eng/Health_standards/tahm/3.03.04_Al.pdf) is at an HI titre of 32 (5 Log2). The hatchlings used experimentally here were found to still have an HI titre around this value at 28 days of age, but these chicks started off far above normal MDA levels. Therefore additional active vaccination is normally required.

For confirmation the antibody titres of the hatchlings were also tested by ELISA, to assure the antibodies measured were directed at AIV H9 HA. A commercial kit was used according to the manufacturer's instructions: ID Screen® Influenza H9 Indirect kit (ID Vet), which is an indirect ELISA. The ELISA scores found, closely matched the pattern of the HI scores. This confirmed that the HI titres detected in the hatchlings originated from antibodies specific for AIV H9 HA.

### Example 2: Preparation of Vaccines for MDA+ Avians

### 2.1. Introduction

Three vaccines were used in the vaccination of the seropositive avians.

The positive control was a classic inactivated whole virus vaccine: Nobilis® Influenza H9N2+ND (MSD Animal Health). This commercial vaccine contains inactivated AIV of subtype H9N2, strain A/Chicken/UAE/415/99 (‘UAE’), and inactivated Newcastle disease virus, strain Clone 30.

The HA proteins of AIV H9N2 strains UDL-01 and UAE have 94% amino acid identity when aligned over their full length.

The NDV component in the inactivated vaccine was not considered to have any significant effect on the efficacy of the AIV vaccination.

Further, two variants of a recombinant HA antigen-based vaccine were used: one version untargeted, and one targeted to CD83 by a fusion to a CD83-scFv. This last version is a recombinant protein for use according to the invention.

### 2.2. Materials and Methods

### 2.2.1. Preparation of HA Antigen Expression Constructs

A mouse hybridoma producing antibodies against chicken CD83 (GenBank acc. nr. XM_040663657.1) was used to obtain the vL and vH chain sequences. Synthetic cDNA containing the vL and vH sequences were joined by (Gly₄Ser)₄ linker peptide sequence and manufactured commercially by Geneart (Thermo Fisher Scientific). The vH-Linker-vL cDNA was then cloned into a D. melanogaster expression vector: pMT-BIP-V5-His™ (Version A, Thermo Fisher Scientific) using the Notl and Xbal restriction sites. This vector provides the D. melanogaster metallothionein (MT) promoter and the D. melanogaster immunoglobulin heavy chain binding protein (BIP) secretion signal, for expression and secretion in S2 cells. Further, multiple cloning sites, a V5 epitope for recombinant protein detection, and a 6×His tag for recombinant protein purification are provided in this plasmid.

The resultant vector named pMT-BIP-CD83-scFv-V5-His was used to insert the ectodomain of an H9 HA gene that lacked the HA gene signal peptide and the TM domain. A 29 amino acid trimerization Foldon sequence was added from the trimeric protein fibritin from bacteriophage T4, using Kpnl and Pacl restriction sites. This plasmid comprised the nucleotide sequence of SEQ ID NO: 14, under the operational control of the MT promoter.

The H9 HA used in this study was synthetically produced incorporating consensus sequence of HA of H9N2 viruses derived from analysis of over 2000 H9 HA sequences from the public databases of G1-like H9 virus lineage using the Minimum Sphere Consensus (MScon) method (Kim et al., 2015, abstracts from German Conference on Bioinformatics, Dortmund, Sep. 27-30, 2015, poster 20: PeerJ PrePrints 3:e1350v1), which is also closely related to the COBRA technique (Giles et al., 2011, Vaccine, vol. 29, p. 3043-3054).

This synthetic HA has 98% amino acid sequence identity to the HA ectodomain of the H9N2 virus of strain UDL-01 (GenBank accession number: ACP50708.1, HA1: aa 19-338 and HA2: aa 339-560), which would qualify it as homologous, and is codon optimised towards S2 cells.

The H9 HA-Foldon antigen without CD83 targeting signal was prepared in a similar way, to provide plasmid pMT-BIP-H9HA-Foldon-V5-His. This plasmid comprised the nucleotide sequence of SEQ ID NO: 15, under the operational control of the MT promoter.

### 2.2.2. Generation and Selection of Recombinant Insect Cells

S2 cells (Thermo Fisher Scientific) were maintained in Schneider's insect medium (Merck GmbH Life Science) supplemented with 10% v/v foetal bovine serum and grown at 28° C. The cells were passaged once a week by centrifuging at 1200 rpm for 10 minutes, and resuspending in fresh complete S2 cell medium.
Recombinant proteins were produced and purified using the Drosophila Expression System (DES®, Life Technologies). In short: the plasmids pMT-BIP-rH9HA-V5-His and pMT-BIP-rH9HA-CD83-scFv-V5-His, were each co-transfected into S2 cells using calcium phosphate transfection. Prior to the transfection, S2 cells at 1×10\hat{ }6/mL had been pre-seeded in 5 mL of complete S2 cell growth medium for 6 to 16 hours at 28° C. A transfection solution was prepared by adding 60 μL 2 M CaCl₂, 32 μg of expression plasmid DNA, 1.5 μg of a hygromycin B resistance plasmid (pCoHYGRO, Life Technologies), and sterile water to bring the total volume up to 500 μL. The transfection solution was slowly added to the equal volume of 2× Hepes buffered saline (HBS) and incubated at room temperature for 30 minutes. The resulting solution was slowly added dropwise to the pre-seeded S2 cells and incubated for 24 hours at 28° C. At 24 hours post transfection, the transfection medium was replaced with fresh complete S2 cell medium and the cells were incubated for 3 more days at 28° C.

Stable S2 transfectants were generated by antibiotic selection: complete growth medium containing hygromycin B at 250 μg/mL was added every week for at least 4 weeks.

Next a single cell clone was obtained via limiting dilution (Zitzmann et al., 2010, Biotechnol. Reports, vol. 19, e00272). In short: 2×10\hat{ }3 S2 transfected cells were mixed with gamma-irradiated 10\hat{ }6 parental S2 cells as feeder cells. 100 μL of this mixture of cells was seeded into each well of 96-well plates. The single clones within each well became clearly visible after 4 weeks of incubation at 28° C. About 10-15 single clones were screened per plasmid construct. The single clones expressing the highest amount of recombinant protein were selected by indirect ELISA for H9 HA protein.

### 2.2.3. Expression and Purification of Recombinant Antigens

The selected transfected S2 cell clones were then cultured at larger scale. In short: single clones expressing high amounts of the HA recombinant proteins were grown in 2 litre roller bottles (Corning) containing 400 mL of Ex-Cell® 420 serum-free medium (Merck GmbH Life Science) for expression and purification. The metallothionein promoter in the plasmids used was induced by adding CuSO₄ to a final concentration of 500 μM. After 4 days post induction, the cell supernatants were harvested via centrifugation at 1200 rpm for 20 minutes and dialysed to remove the excess copper ions. In total, about 2 litres of protein expression supernatants were collected and filtered through 0.22 μM filter stericup (Merck GmbH Life Science) before purification.
The use of the His tag allowed purification of the recombinant proteins by metal-affinity column chromatography. In short: the dialysed and filtered supernatants containing recombinant proteins were loaded on 10 mL Profinity™ IMAC uncharged resin column (Bio-Rad) and washed with 5 column volumes of wash buffer. Copper-bound proteins were then eluted with elution buffers containing increasing concentration of imidazole (25, 50, 100 or 500 mM). The purified proteins were analysed using SDS-PAGE on 10% PAA gels, followed by Coomassie Blue staining. The protein fractions were combined and concentrated using 15 mL Amicon Ultra-15™ Centrifugal Filter column (3 kDa MWCO, Merck GmbH Life Science) by centrifuging at 4600 rpm for 30 minutes. The concentration of the purified proteins was determined using a Pierce BCA Protein Assay Kit™ (Life Technologies) according to the manufacturer's instructions.

The H9 HA activity of the recombinant proteins produced was confirmed using a haemagglutination assay. Briefly, 35 μg of the recombinant protein was serially diluted 2-fold in PBS in V-bottom 96-well plates. Chicken red blood cells were diluted to 1% in PBS and added to each well. The plates were then incubated at 4° C. for 1 hour, tipped 90° in a biosafety cabinet to visualise haemagglutination, and scored.

### 2.2.4. Preparation of Vaccine Emulsions

The recombinant HA antigen vaccines were formulated as water-in-oil emulsions, with light liquid paraffin oil (Marcol® 52) as adjuvant, and contained Polysorbate 80 (Tween® 80) and Sorbitan mono-oleate (Span® 80) as emulsifiers. The water: oil weight-ratio of the vaccines was 45:55. All vaccines were stored at 4° C. until use.

The recombinant HA vaccines contained per dose of 0.2 ml: 35 μg of the untargeted HA antigen, or 49 μg of the targeted antigen. This difference was to provide equimolar amounts, compensating for the addition of the scFv

### Example 3: Vaccination of Seropositive Birds

### 3.1. Introduction

As the protection against AIV infection and disease is essentially serologically determined, and the main AIV-neutralising antibodies are those against the HA antigen, therefore serological testing for anti-HA antibody development, i.e. HI titre determination, is an excellent predictor of in vivo protection from AIV.

The hatchlings generated as described in Example 1, were used in a vaccination experiment: one group was vaccinated at day 1, these had a very high average MDA HI titre of 588 (9.2 Log2), called the MDA++ group. One other group was only vaccinated at day 14 of age, when MDA levels had decreased somewhat, these had a medium average MDA HI titre of 181 (7.5 Log2), called the MDA+ group.

This approach allowed the testing and comparison of a ‘worst case’ and an ‘average case’ of antibody interference, respectively, on the efficacy of vaccination with targeted or untargeted HA antigen. For comparison a classic inactivated H9N2 vaccine was included. Also a non-vaccinated group of chicks was included in the study to follow the natural decline of the anti-AIV H9 HA MDA levels.

### 3.2. Materials and Methods

### 3.2.1. Animals, Sampling and Vaccinations

The AIV H9 HA MDA positive chicks used, were obtained as described in Example 1. The vaccines used were as described in Example 2.
To avoid incoming environmental pathogens, the birds were housed in positive pressure isolation rooms with high-efficiency particulate air (HEPA) filtered air inflow.

After hatching, only chicks that appeared healthy and normal were used. These were assigned to the groups as they came to hand, and were individually numbered. Daily clinical observations were made to monitor health and performance. Each test group had 10 animals.

All vaccines were at ambient temperature at use, and were mixed strongly just before use to ensure homogeneity.

All chicks received only a single vaccination, either on day 1 or on day 14 of age. The administration was via the standard route for these types of vaccines: subcutaneous (sc). A volume of 0.25 ml/dose was used for the Nobilis® vaccine as that is the registered dose; for the recombinant HA antigen vaccines, 0.2 ml/dose was given.

Nobilis Influenza H9N2+ND vaccine was given at day 1 to MDA++. The H9HA-Foldon and the H9HA-Foldon-CD83-scFv vaccines were given both to the ‘MDA++’ chicks at day 1, and to the ‘MDA+’ chicks that were then 14 days of age.

Blood samples were collected once every week up to week 6 post start of the experiment, and once every two weeks for weeks 8, 10 and 12 post start, to determine the serological responses induced by the vaccinations.

Blood samples at days 1 and 7 were collected after euthanisation; samples from day 14 onwards were taken from the wing vein. Volumes collected were 2-3 ml, as permitted by the weight of the animals. Blood samples were left at ambient temperature to clot, and serum was separated by centrifugation. The serum samples were heat-inactivated for 30 min. at 56° C., and stored at −20° C. until use.

### 3.3. Results

The results of the HI titrations with the serum samples taken during this experiment from the MDA++ and the MDA+ chicks, are presented in FIGS. 3 and 4 respectively.
The non-vaccinated controls showed a level of HI titre, and a pattern of degradation, as described in Example 1, and FIG. 2.
The positive controls were MDA++ chicks receiving whole inactivated virus vaccine (‘Nobilis Influenza H9N2+ND’) at day one of age. In spite of this vaccination, their HI titres steadily declined, and no vaccination response could be detected. This is remarkable, as the MDA and the HA antigen in the classic vaccine were heterologous: the MDA were induced against an HA antigen that closely resembled the H9 HA of strain UDL-01, while the Nobilis vaccine contained a heterologous H9 HA antigen, namely that from strain UAE, which has 94% amino acid identity to the UDL-01 H9 HA protein. Consequently one would expect a lesser level of antibody interference because of this difference between the HA antigens. Apparently however, the HI levels in the MDA++ chicks were so high that they even interfered with the efficacy of a heterologous H9 HA vaccine.
The vaccinations with targeted (‘H9HA Foldon-CD83-scFv’) and untargeted (‘H9HA Foldon’) HA antigen showed spectacular differences in the HI titres they induced, both in the MDA++ and in the MDA+ chicks.

The HI titres in the chicks vaccinated with untargeted HA antigen steadily decreased and did not show any significant rise in HI titre at any time point after vaccination, neither in the MDA++ nor in the MDA+ chicks.

However the targeted HA antigen induced very high HI titres. In the MDA++ group there was an initial decline from the very high starting value (9.7 Log2), but the HI titres showed a strong and steady increase after that: visible from 4 weeks post vaccination (p.v.), reaching significance at 5 weeks p.v., and strongly increasing to 9.7 log2 at 12 weeks p.v. This demonstrates that this vaccine can be applied at day 1 of age, even in the context of very high levels of homologous MDA, and is still capable of inducing a strong protection against AIV infection and disease.

In the MDA+ group the HI titres from the targeted HA vaccine showed a rapid induction of high HI titres, already from 1 week after vaccination. The HI titre reached an average of 1835 (10.8 Log2), from 4 weeks after vaccination.

In both test groups, the targeted vaccine was the only one capable of inducing significantly increased HI titres. Also, the lowest HI titre measured in the targeted vaccine groups was 6.2 and 6.9 Log2 in the MDA++ and MDA+ groups respectively. This indicates that all chicks receiving this type of vaccine, remained well above the 5 Log2 threshold for protection, for the duration of the experiment.

This rapid onset of immunity, and long duration, perfectly compensate for the drop-off in MDA level, without leaving a gap in the protection.

Again indirect ELISA was performed on the sera, which confirmed that all antibodies were H9 HA specific.

### Example 4: Targeting of Non-HA Antigens

Experiments essentially similar to those described above are in preparation for recombinant proteins for use according to the invention, but comprising another antigen than AIV HA. These are: AIV HN, NDV F, NDV HN, IBDV VP2, and IBV spike. In short: hens can be vaccinated with a suitable vaccine against one of these pathogens: AIV, NDV, IBDV, or IBV; such vaccines are generally available.

The hens can be given 2 or 3 vaccinations, starting before onset of lay, and continuing during their laying period. The specific antibody titres reached in the hens can be checked to be sufficiently high. Then eggs can be collected and hatched, and the chicks can be checked for having sufficiently high MDA levels for the pathogen to be studied.

Vaccines can be prepared comprising a recombinant protein for use according to the invention, as described above, for example by constructing an expression plasmid, comprising a nucleotide sequence encoding one of the antigens to be tested. Also a binding domain will be comprised, e.g. an scFv, directed at an avian APC's surface protein, such as CD83, CD11c, or Dec-205. Similar constructs but without a binding domain can be prepared to serve as control, to assess the effect of the targeting of the antigen to the APC.

The plasmids can be transfected into S2 cells as described, these can be selected, amplified, and used to express the antigen (with or without targeting signal). Next recombinant protein can be harvested.

An example of an CD83-scFv is a peptide comprising the amino acid sequence of SEQ ID NO: 2. An example of an scFv specific for CD11c or Dec-205, is a peptide comprising an amino acid sequence as presented in SEQ ID NO: 16 or 17, respectively.

Examples of antigens to be expressed comprise an amino acid sequence selected from:
for AIV H5 HA: SEQ ID NO: 4;
for AIV H7 HA: SEQ ID NO: 5;
for NDV F: SEQ ID NO: 18;
for NDV HN: SEQ ID NO: 19;
for IBDV VP2: SEQ ID NO: 20, and
for IBV spike: SEQ ID NO: 21.
   The corresponding nucleic acids, encoding these antigens are preferably codon optimised towards the codon usage table of S2 cells. In the expression construct, additional elements can be added when desired, such as a signal sequence, a linker, and one or more tags, in order to facilitate expression, secretion, and purification.
   Next, the chicks with the specific MDAs will be vaccinated with these recombinant proteins, and their serology will be monitored over time.

Because for these pathogens the levels of specific antibodies are known that correlate with in vivo protection, therefore serological testing of antibody levels at various times after vaccination suffices to get a good impression on the efficacy of targeted vaccination in avians seropositive for the antigen from these pathogens.

The H5 HA sequence of SEQ ID NO: 4, was derived from the HA of AIV isolate:
A/duck/Egypt/SS19/2017, H5N8, GenBank acc.nr. AXY66755.1. Selected were 511 aa of the HA ectodomain: HA1: 17-340 and HA2: 346-530. The polybasic cleavage sequence was modified: from PLR to PQG, and the number of arginines was reduced.
The H7 HA sequence of SEQ ID NO: 5, was derived from the HA of AIV isolate:
A/chicken/Jiangxi/JX4/2017, H7N9, GenBank acc.nr. ARG44105.1. Selected was the HA ectodomain of 507 amino acids: HA1: 19-339 and HA2: 1-186, with a modified polybasic cleavage sequence: from PKR to PKG.
The NDV F sequence of SEQ ID NO: 18, is the consensus sequence from over 1200 F amino acid sequences from avian avulavirus 1 sequences in public databases, using the MScon technique as described herein. The consensus F protein has 98.5% amino acid similarity to the closest natural relative: avian orthoavulavirus 1 F protein, GenBank acc. nr. AHX74055.1. The F protein ectodomain was selected from aa. 31-500.
The NDV HN sequence of SEQ ID NO: 19, is a consensus sequence, starting from the HN protein from Avian orthoavulavirus 1 of GenBank acc. nr. AXK59828.1, combined with a number of HN sequences from the public databases, using the MScon technique as described herein. Amino acids 47-571 from the HN were selected.
The IBDV VP2 protein of SEQ ID NO: 20, represents aa 9-452 from IBDV VP2 protein of GenBank acc. nr. AMA19770.1.
The IBV spike protein of SEQ ID NO: 21, represents aa 1-1096 from IBV spike protein of GenBank acc. nr. ARS22410.1. The spike protein was stabilised by making two amino acid substitutions: Q859P and L860P.

### LEGEND TO THE FIGURES

FIG. 1

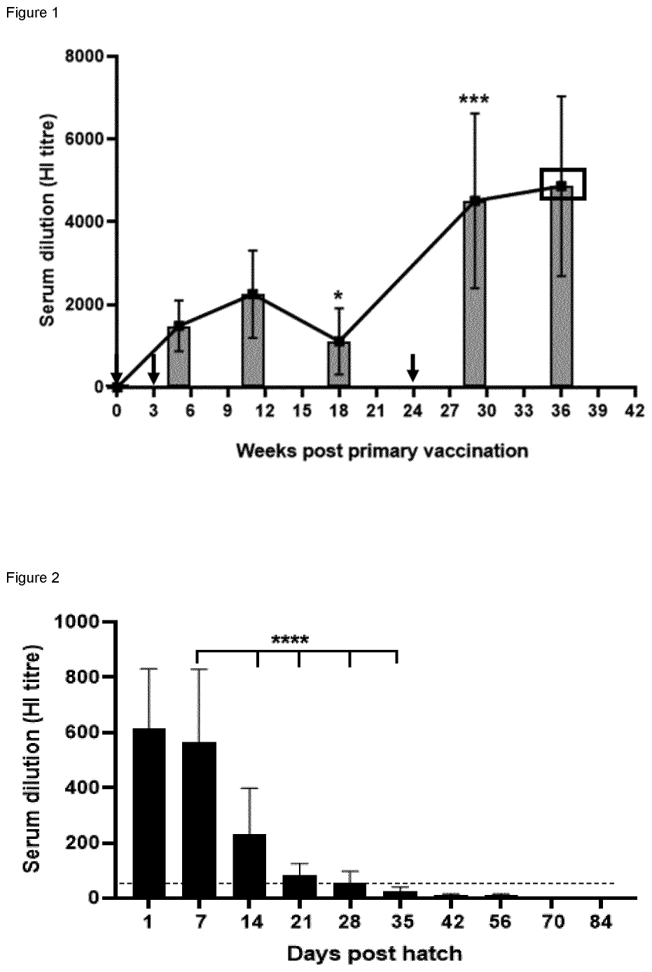

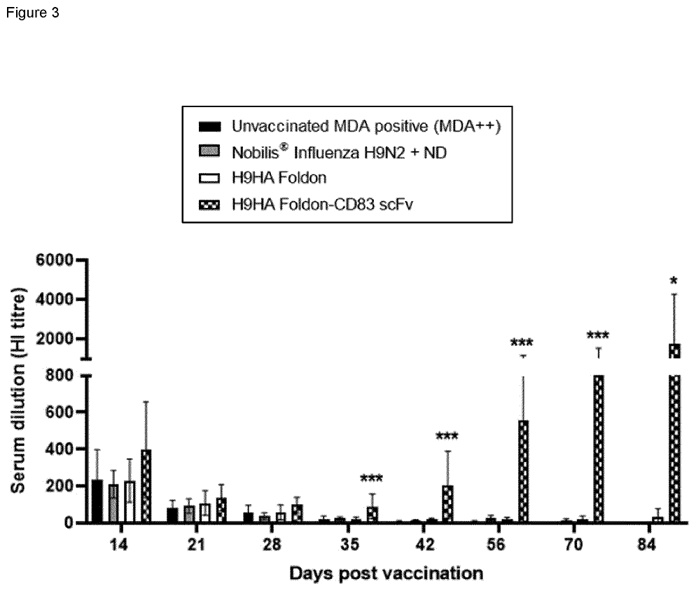

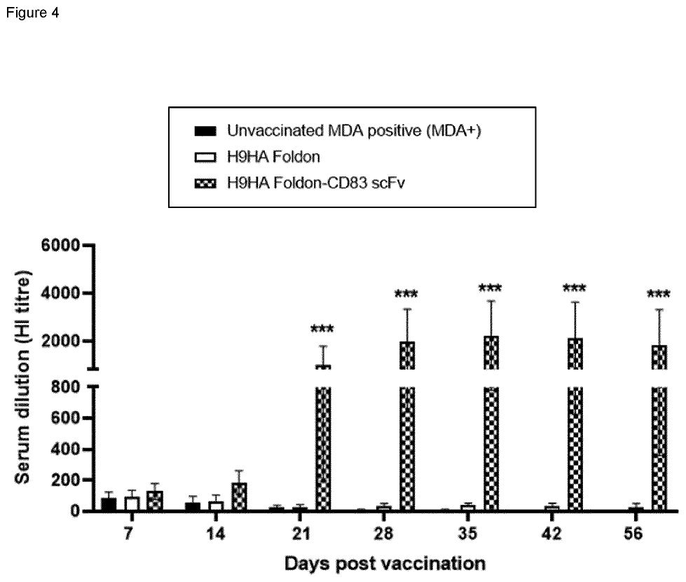

Presentation of the results of antibody titres in hyper-immunised mother hens, in order to generate AIV MDA+ offspring. Details are described in Example 1.

The vertical axis presents the average (n=10) HI titres measured by HI assay in the serum of mother hens, after immunisation with inactivated-adjuvated AIV H9N2 virus vaccine (UDL 01/08). The time points are indicated on the horizontal axis, in weeks post start of the experiment (day 0=17 weeks of age). Vaccinations are indicated by arrows, at 0, 3, and 24 weeks post start.

Fertilized eggs were collected after 36 weeks post start; the box indicates the average (n=10) HI titre in the hen's serum at the time of laying the eggs that were used in the follow up experiment: HI=12 Log2 (4096).

Data are presented as mean (columns) and standard deviation (error bars). The asterisks represent a significant difference between the HI antibody titres at 11 and 18 weeks post start of the experiment, and at 18 and 29 weeks post start, whereby: *=p<0.05, and ***=p<0.001.

FIG. 2

Presentation of the results of the MDA derived HI titres in unvaccinated offspring from the triple vaccinated hens. Details are described in Example 1.

Anti-H9 HA MDA titres were measured by HI assay in serum samples from day 1, 7, 14, 21, 28, 35, 42, 56, 70 and 84 post hatch. HI titres are expressed as the reciprocal of the highest dilution of the serum causing the total inhibition of 4 HA units of virus hemagglutination activity. Data are presented as mean±SD, and were analysed by one-way ANOVA followed by Tukey's multiple comparison test. Statistically significant differences are shown as: ****=p<0.0001.

The dotted horizontal line indicates the minimal protective level of the HI titre of 32 (5 Log2).

FIG. 3

Presentation of the results of the HI titres in the chicks vaccinated at day 1 of age, having high levels of MDA (MDA++). Details are described in Example 3.

The vertical axis indicates HI titres, and the horizontal axis the days post vaccination. NB: There is a gap in the vertical axis to be able to display the very high HI titres found.

Groups of MDA++ chicks (n=10) were vaccinated at day 1 of age with one of three vaccines: whole inactivated virus vaccine (‘Nobilis Influenza H9N2+ND’); untargeted HA antigen (‘H9HA Foldon’); or CD83 targeted HA antigen (‘H9HA Foldon-CD83-scFv’). As controls, one group of MDA++ chicks remained unvaccinated.

Anti-H9 HA antibody titres were measured by HI assay, using the UDL-01 virus in the HI assay.

The data are presented as mean (columns) and SD (error bars). Statistically significant differences are indicated with asterisks, whereby: ***=p<0.001 and *=p<0.1.

FIG. 4

Presentation of the results of the HI titres of the chicks vaccinated at day 14 of age, having medium levels of MDA (MDA+). Details are described in Example 3.

Similar presentation as for FIG. 3, except that there was no group vaccinated with the Nobilis vaccine.

1-12. (canceled)

13. A method for protecting an avian against a pathogen, the method comprising the step of administering to said avian a vaccine comprising a pharmaceutically acceptable carrier and a recombinant protein comprising an antigen and a binding domain that is capable of binding to a cell surface protein on an avian antigen-presenting cell (APC), whereby the antigen that is comprised in said vaccine was derived from said pathogen, and whereby said avian possess antibodies reactive with said antigen.

14. The method of claim 13, wherein the avian APC is a dendritic cell.

15. The method of claim 13, wherein the cell surface protein is CD83.

16. The method of claim 13, wherein the binding domain is a single-chain variable fragment (scFv).

17. The method of claim 13, wherein the antigen is selected from: infectious bursal disease virus (IBDV) viral protein 2 (VP2), Newcastle disease virus (NDV) fusion (F) protein, NDV hemagglutinin-neuraminidase (HN) protein, infectious bronchitis virus (IBV) spike protein, avian Influenza virus (AIV) haemagglutinin (HA) protein, and AIV neuraminidase (NA) protein.

18. The method of claim 13, wherein the antigen comprises the amino acid sequence selected from: SEQ ID NO's: 7, 8, and 9.
